(19) United States
(12) Patent Application Publication
(10) Pub. No.: US 2011/0105413 A1
(43) Pub. Date: May 5, 2011

(54) POLYMERIC SYSTEMS CONTAINING INTRACELLULAR RELEASABLE DISULFIDE LINKER FOR THE DELIVERY OF OLIGONUCLEOTIDES

(75) Inventors: Hong Zhao, Edison, NJ (US); Prasanna Reddy, Somerset, NJ (US)

(73) Assignee: ENZON PHARMACEUTICALS, INC., Bridgewater, NJ (US)

(21) Appl. No.: 12/994,266

(22) PCT Filed: May 22, 2009
(86) PCT No.: PCT/US2009/044953

§ 371 Date: Nov. 23, 2010

Related U.S. Application Data
(60) Provisional application No. 61/055,950, filed on May 23, 2008.
(60) Provisional application No. 61/055,869, filed on May 23, 2008.
(60) Provisional application No. 61/106,578, filed on Oct. 19, 2008.
(60) Provisional application No. 61/106,576, filed on Oct. 19, 2008.

(51) Int. Cl. A61K 38/02; A61K 31/7088; A61K 31/713; C12N 5/071; C12N 5/09; C08B 37/00; A61P 35/00

(52) U.S. Cl. 514/20.9; 514/44 R; 514/44 A; 435/375; 536/53

(57) ABSTRACT

The present invention provides polymeric prodrugs including an intracellular releasable disulfide linker for the delivery of oligonucleotides. Methods of making the compounds as well as methods of delivering nucleic acids to tumor cells in a mammal using the same are also provided.

### CROSS-REFERENCE TO RELATED APPLICATION

This application claims the benefit of priority from U.S. Provisional Patent Application Ser. Nos. 61/055,950 filed May 23, 2008, 61/055,869 filed May 23, 2008, 61/106,578 filed Oct. 19, 2008, and 61/106,579 filed Oct. 19, 2008, the contents of each of which are incorporated herein by reference.

### BACKGROUND OF THE INVENTION

Gene-based therapy is a powerful tool in the treatment of disease because a therapeutic gene can selectively modulate gene expression associated with disease and minimize side effects which may incur when other therapeutic approaches are used.

A therapy based on locked Nucleic Acid (LNA) antisense oligonucleotide, a new generation of RNA antagonist, has been proposed. Each LNA monomer contains a methylene bridge between the 2′-oxygen and 4′-carbon of the ribose sugar. This fixes the LNA residue in a favorable RNA-like conformation and enables LNA oligonucleotides to have higher affinity, specificity, and resistance against degradation compared with other art-known oligonucleotides. It has been shown that LNA oligonucleotide inhibits target gene expression in vitro (at sub-nanomolar level). While LNA oligonucleotides have improved therapeutic activity compared to other art-known nucleic acids, it is still needed to further improve the pharmacokinetic profile and fast clearance from circulation and limited activity in vivo of LNA oligonucleotides. There continues to be a need to provide improved systems and methods for the delivery of LNA oligonucleotides as well as other art-known nucleic acid molecules. The present invention addresses this need.

### SUMMARY OF THE INVENTION

In order to overcome the above problems and improve the technology for the delivery of oligonucleotides, the present invention provides new polymeric delivery systems containing an intracellularly releasable linker.

In one aspect of the present invention, there are provided methods of delivering oligonucleotides to tumor cells in a mammal. The methods include administering to the mammal having tumor cells a compound of Formula (I):

R₁—{Z₁}ₘ

or a pharmaceutically acceptable salt thereof,

wherein

R₁ is a substantially non-antigenic water-soluble polymer;

each Z₁ is the same or different and selected from among:

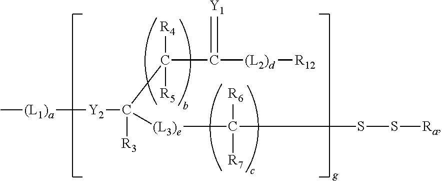

-(L₄)ₐ₁-R_{b}; and

-(L₄)ₐ₂-R_{c},

Y₁, in each occurrence, is independently S or O;

Y₂, in each occurrence, is independently NR₁₃;

Rₐ, in each occurrence, is the same or a different oligonucleotide;

each of L₁₋₄, in each occurrence, is the same or a different bifunctional linker;

R_{b}, in each occurrence, is a folic acid;

R_{c}, in each occurrence, is the same or a different diagnostic agent;

each of R₃₋₇ is independently selected from among hydrogen, C₁₋₆ alkyls, C₂₋₆ alkenyl, C₂₋₆ alkynyl, C₃₋₁₉ branched alkyl, C₃₋₈ cycloalkyl, and C₁₋₆ alkoxy;

R₁₃, in each occurrence, is independently selected from among hydrogen, C₁₋₆ alkyls, C₂₋₆ alkenyl, C₂₋₆ alkynyl, C₃₋₁₉ branched alkyl, and C₃₋₈ cycloalkyl;

R₁₂, in each occurrence, is independently selected from among hydrogen, hydroxyl, C₁₋₆ alkyls, C₂₋₆ alkenyl, C₂₋₆ alkynyl, C₃₋₁₉ branched alkyl, C₃₋₈ cycloalkyl, and C₁₋₆ alkoxy;

each of (a) and (d) is independently 0, 1, 2 or 3;

each of (a1) and (a2) is independently 0, 1, 2 or 3;

each (b) is independently 0, 1, 2, or 3;

each (c) is independently 0, 1, 2 or 3;

each (e) is independently 0 or 1;

each (g) is independently 0 or 1; and

(m) is a positive integer from about 2 to about 32,

provided that (a) and (g) are not simultaneously zero and further provided that one or more of Z₁ contain an oligonucleotide.

In another aspect, the present invention provides a method of inhibiting a gene expression in a mammal having prostate or cervical cancer cells.

In one embodiment, the compound of Formula (I) employed in the method described herein is:

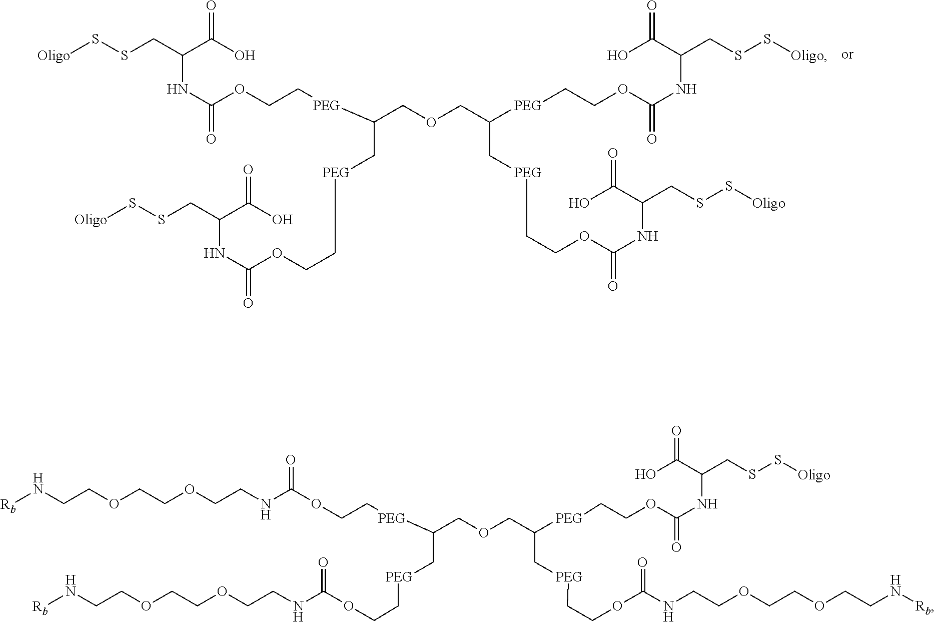

or a pharmaceutically acceptable salt thereof,

wherein

PEG is a polyethylene glycol and the polymeric portion of the compound has the total number average molecular weight of from about 5,000 to about 25,000 daltons or from about 20,000 to about 45,000 daltons;

R_{b} is

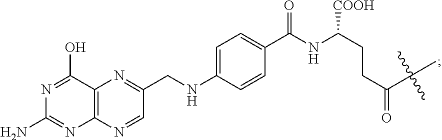

and

Oligo is an oligonucleotide of from about 8 to 30 nucleotides.

In a further aspect of the invention, there are provided methods of inhibiting a gene expression in a mammal for the treatment of various diseases (i.e. prostate or cervical cancer).

The present invention also provides methods of making the compounds described herein.

One advantage of the polymeric transport systems described herein is that the releasable PEG-linker technology provides a method for in vivo administration of therapeutic oligonucleotides including LNA. This selective delivery technology allows enhanced therapeutic efficacy of LNA and decrease in toxicity.

Another advantage is that the releasable delivery systems described herein allow for modulating of the pharmacokinetic properties of oligonucleotides. The release site of therapeutic oligonucleotides from the polymeric conjugates can be selectively targeted via EPR effect and a targeting group such as a folic acid. The oligonucleotides such as LNA attached to the polymers described herein can be released at a targeted area, such as cancer cells, thus allowing the artisan to achieve a desired bioavailability of therapeutic oligonucleotides at a targeted area. In addition, the site of release of the oligonucleotides can be modified, i.e., to release oligonucleotides in different compartments of the cells. Thus, the polymeric delivery systems described herein allow sufficient amounts of the therapeutic oligonucleotides including LNA to be selectively available at the desired target area, e.g., cytoplasm, micropinosome and endosome. The spatial modifications can be advantageous for treatment of disease. The methods described herein provide an approach for the delivery and improved efficacy of oligonucleotides (e.g., LNA oligonucleotide, siRNA) in vivo.

A further advantage of the present invention is that the conjugates described herein allow cellular uptake and specific mRNA downregulation in cancer cells in the absence of transfection agents. This is a significant advantage over prior art technologies and thus significantly simplifies treatment regimens, i.e., the in vivo administration of oligonucleotide drugs. This technology can be applied to the in vivo administration of therapeutic oligonucleotides.

The polymeric compounds are stable under buffer conditions and the oligonucleotides or other therapeutic agents are not prematurely excreted from the body.

Further advantages will be apparent from the following description and drawings.

For purposes of the present invention, the term “residue” shall be understood to mean that portion of a compound to which it refers, i.e., PEG, oligonucleotide, etc. that remains after it has undergone a substitution reaction with another compound.

For purposes of the present invention, the term “polymeric residue” or “PEG residue” shall each be understood to mean that portion of the polymer or PEG which remains after it has undergone a reaction with other compounds, moieties, etc.

For purposes of the present invention, the term “alkyl” as used herein refers to a saturated aliphatic hydrocarbon, including straight-chain, branched-chain, and cyclic alkyl groups. The term “alkyl” also includes alkyl-thio-alkyl, alkoxyalkyl, cycloalkylalkyl, heterocycloalkyl, C₁₋₆ hydrocarbonyl, groups. Preferably, the alkyl group has 1 to 12 carbons. More preferably, it is a lower alkyl of from about 1 to 7 carbons, yet more preferably about 1 to 4 carbons. The alkyl group can be substituted or unsubstituted. When substituted, the substituted group(s) preferably includes halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “substituted” as used herein refers to adding or replacing one or more atoms contained within a functional group or compound with one of the moieties from the group of halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

The term “alkenyl” as used herein refers to groups containing at least one carbon-carbon double bond, including straight-chain, branched-chain, and cyclic groups. Preferably, the alkenyl group has about 2 to 12 carbons. More preferably, it is a lower alkenyl of from about 2 to 7 carbons, yet more preferably about 2 to 4 carbons. The alkenyl group can be substituted or unsubstituted. When substituted, the substituted group(s) preferably includes halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

The term “alkynyl” as used herein refers to groups containing at least one carbon-carbon triple bond, including straight-chain, branched-chain, and cyclic groups. Preferably, the alkynyl group has about 2 to 12 carbons. More preferably, it is a lower alkynyl of from about 2 to 7 carbons, yet more preferably about 2 to 4 carbons. The alkynyl group can be substituted or unsubstituted. When substituted, the substituted group(s) preferably includes halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups. Examples of “alkynyl” include propargyl, propyne, and 3-hexyne.

The term “aryl” as used herein refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

The term “cycloalkyl” as used herein refers to a C₃₋₈ cyclic hydrocarbon. Examples of cycloalkyl include cyclopropyl, cyclobutyl, cyclopentyl, cyclohexyl, cycloheptyl and cyclooctyl.

The term “cycloalkenyl” as used herein refers to a C₃₋₈ cyclic hydrocarbon containing at least one carbon-carbon double bond. Examples of cycloalkenyl include cyclopentenyl, cyclopentadienyl, cyclohexenyl, 1,3-cyclohexadienyl, cycloheptenyl, cycloheptatrienyl, and cyclooctenyl.

The term “cycloalkylalkyl” as used herein refers to an alkyl group substituted with a C₃₋₈ cycloalkyl group. Examples of cycloalkylalkyl groups include cyclopropylmethyl and cyclopentylethyl.

The term “alkoxy” as used herein refers to an alkyl group of the indicated number of carbon atoms attached to the parent molecular moiety through an oxygen bridge. Examples of alkoxy groups include, for example, methoxy, ethoxy, propoxy and isopropoxy.

An “alkylaryl” group as used herein refers to an aryl group substituted with an alkyl group.

An “aralkyl” group as used herein refers to an alkyl group substituted with an aryl group.

The term “alkoxyalkyl” group as used herein refers to an alkyl group substituted with an alkoxy group.

The term “alkyl-thio-alkyl” as used herein refers to an alkyl-5-alkyl thioether, for example, methylthiomethyl or methylthioethyl.

The term “amino” as used herein refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups, respectively.

The term “alkylcarbonyl” as used herein refers to a carbonyl group substituted with alkyl group.

The terms “halogen” or “halo” as used herein refer to fluorine, chlorine, bromine, and iodine.

The term “heterocycloalkyl” as used herein refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrrolidinyl.

The term “heteroaryl” as used herein refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

The term “heteroatom” as used herein refers to nitrogen, oxygen, and sulfur.

In some embodiments, substituted alkyls include carboxyalkyls, aminoalkyls, dialkylaminos, hydroxyalkyls and mercaptoalkyls; substituted alkenyls include carboxyalkenyls, aminoalkenyls, dialkenylaminos, hydroxyalkenyls and mercaptoalkenyls; substituted alkynyls include carboxyalkynyls, aminoalkynyls, dialkynylaminos, hydroxyalkynyls and mercaptoalkynyls; substituted cycloalkyls include moieties such as 4-chlorocyclohexyl; aryls include moieties such as napthyl; substituted aryls include moieties such as 3-bromo phenyl; aralkyls include moieties such as tolyl; heteroalkyls include moieties such as ethylthiophene; substituted heteroalkyls include moieties such as 3-methoxy-thiophene; alkoxy includes moieties such as methoxy; and phenoxy includes moieties such as 3-nitrophenoxy. Halo shall be understood to include fluoro, chloro, iodo and bromo.

For purposes of the present invention, “positive integer” shall be understood to include an integer equal to or greater than 1 and as will be understood by those of ordinary skill to be within the realm of reasonableness by the artisan of ordinary skill, i.e., preferably from 1 to about 10, more preferably 1 or 2 in some embodiments.

For purposes of the present invention, the terms, “nucleic acid” or “nucleotide” apply to deoxyribonucleic acid (“DNA”) and ribonucleic acid, (“RNA”), whether single-stranded or double-stranded, unless otherwise specified, and any chemical modifications thereof.

For purposes of the present invention, the term “linked” shall be understood to include covalent (preferably) or noncovalent attachment of one group to another, i.e., as a result of a chemical reaction.

The terms “effective amounts” and “sufficient amounts” for purposes of the present invention shall mean an amount which achieves a desired effect or therapeutic effect as such effect is understood by those of ordinary skill in the art.

For purposes of the present invention, the term “therapeutic oligonucleotide” refers to an oligonucleotide used as a pharmaceutical or diagnostic agent.

For purposes of the present invention, “modulation of gene expression” shall be understood as broadly including down-regulation or up-regulation of any types of genes, preferably associated with cancer and inflammation, compared to a gene expression observed in the absence of the treatment with the compounds described herein, regardless of the route of administration.

For purposes of the present invention, “inhibition of gene expression” of a target gene shall be understood to mean that mRNA expression or protein translated are reduced or attenuated when compared to that observed in the absence of the treatment with the compound described herein. Suitable assays include, e.g., examination of protein or mRNA levels using techniques known to those of skill in the art such as dot blots, northern blots, in situ hybridization, ELISA, immunoprecipitation, enzyme function, as well as phenotypic assays known to those of skill in the art. The treated conditions can be confirmed by, for example, decrease in mRNA levels in cells, preferably cancer cells or tissues.

Broadly speaking, successful inhibition or treatment shall be deemed to occur when the desired response is obtained. For example, successful inhibition or treatment can be defined by obtaining e.g., 10% or higher (i.e., 20% 30%, 40%) downregulation of genes associated with tumor growth inhibition. Alternatively, successful treatment can be defined by obtaining at least 20% or preferably 30%, more preferably 40% or higher (i.e., 50% or 80%) decrease in oncogene mRNA levels in cancer cells or tissues, including other clinical markers contemplated by the artisan in the field, when compared to that observed in the absence of the treatment with the compound described herein.

Further, the use of singular terms for convenience in description is in no way intended to be so limiting. Thus, for example, reference to a composition comprising an enzyme refers to one or more molecules of that enzyme. It is also to be understood that this invention is not limited to the particular configurations, process steps, and materials disclosed herein as such configurations, process steps, and materials may vary somewhat.

It is also to be understood that the terminology employed herein is used for the purpose of describing particular embodiments only and is not intended to be limiting, since the scope of the present invention will be limited by the appended claims and equivalents thereof.

### BRIEF DESCRIPTION OF THE DRAWINGS

FIG. 1 schematically illustrates synthesis of compound 1 described in Example 7.

FIG. 2 schematically illustrates synthesis of compound 10 described in Examples 8-13.

FIG. 3 schematically illustrates synthesis of compound 18 described in Examples 14-18.

FIG. 4 schematically illustrates synthesis of compound 25 described in Examples 19-23.

FIG. 5 schematically illustrates synthesis of compound 30 described in Examples 25-27.

FIG. 6 schematically illustrates synthesis of compound 35 described in Examples 28-29.

FIG. 7 shows cellular uptake of PEG-LNA conjugates described in Examples 32.

FIG. 8 shows receptor-specific cellular uptake of PEG-LNA conjugates described in Example 32.

FIG. 9 shows in vitro efficacy of PEG-LNA conjugates described in Example 33.

FIG. 10 shows in vivo efficacy and biodistribution of Folate-PEG-LNA conjugates described in Example 34.

FIG. 11 shows biodistribution of PEG-LNA conjugates described in Example 35.

FIG. 12 shows in vivo efficacy of PEG-LNA conjugates described in Example 36.

FIG. 13 shows in vivo efficacy of PEG-LNA conjugates described in Example 37.

For ease of the description and not limitation, multi-arm PEG (e.g., four-arm PEG) is described as “PEG” in the figures.

### DETAILED DESCRIPTION OF THE INVENTION

### A. Overview

In one aspect of the present invention, there are provided methods of delivering oligonucleotides to tumor cells in a mammal in need thereof. The method includes administering to the mammal having tumor cells a compound of Formula (I):

R₁—{Z₁}ₘ

or a pharmaceutically acceptable salt thereof,

wherein

R₁ is a substantially non-antigenic water-soluble polymer;

each Z₁ is the same or different and selected from among

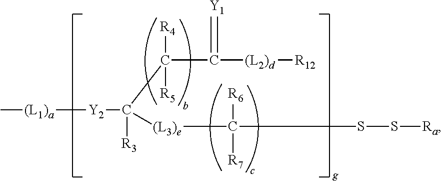

-(L₄)ₐ₁-R_{b}; and

-(L₄)ₐ₂-R_{c},

Y₁, in each occurrence, is independently S or O, preferably O;

Y₂, in each occurrence, is independently NR₁₃, preferably NH;

Rₐ, in each occurrence, is the same or a different oligonucleotide;

each of L₁₋₄, in each occurrence, is the same or a different bifunctional linker;

R_{b}, in each occurrence, is a folic acid;

R_{c}, in each occurrence, is the same or a different diagnostic agent;

each of R₃₋₇ is independently selected from among hydrogen, C₁₋₆ alkyls, C₂₋₆ alkenyl, C₂₋₆ alkynyl, C₃₋₁₉ branched alkyl, C₃₋₈ cycloalkyl, and C₁₋₆ alkoxy;

R₁₃, in each occurrence, is independently selected from among hydrogen, C₁₋₆ alkyls, C₂₋₆ alkenyl, C₂₋₆ alkynyl, C₃₋₁₉ branched alkyl, and C₃₋₈ cycloalkyl;

R₁₂, in each occurrence, is independently selected from among hydrogen, hydroxyl, C₁₋₆ alkyls, C₂₋₆ alkenyl, C₂₋₆ alkynyl, C₃₋₁₉ branched alkyl, C₃₋₈ cycloalkyl and C₁₋₆ alkoxy;

each of (a) and (d) is independently zero, 1, 2, or 3, and preferably 0;

each of (a1) and (a2) is independently zero, 1, 2, or 3, and preferably 1;

each (b) is independently zero, 1, 2, or 3, and preferably 0;

each (c) is independently zero, 1, 2, or 3, and preferably 1;

each (e) is independently zero or one, and preferably 0;

each (g) is independently zero or one, and preferably 1; and

(m) is a positive integer from about 2 to about 32 (e.g., 2, 4, 6, 8, 16, 32),

provided that (a) and (g) are not simultaneously zero and provided that one or more of Z₁ contain an oligonucleotide.

In another aspect, compounds of Formula (I) are provided:

R₁—{Z₁}ₘ

or a pharmaceutically acceptable salt thereof,

wherein

R₁ is a substantially non-antigenic water-soluble polymer;

each Zᵢ is the same or different and selected from among

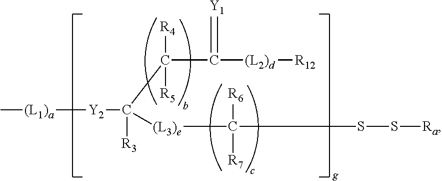

-(L₄)ₐ₁-R_{b}; and

-(L₄)ₐ₂-R_{c}

Y₁, in each occurrence, is independently S or O, preferably O;

Y₂, in each occurrence, is independently NR₁₃, preferably NH;

Rₐ, in each occurrence, is the same or a different oligonucleotide;

each of L₁₋₄, in each occurrence, is the same or a different bifunctional linker;

R_{b}, in each occurrence, is a folic acid;

R_{c}, in each occurrence, is the same or a different diagnostic agent;

each of R₃₋₇ is independently selected from among hydrogen, C₁₋₆ alkyls, C₂₋₆ alkenyl, C₂₋₆ alkynyl, C₃₋₁₉ branched alkyl, C₃₋₈ cycloalkyl, and C₁₋₆ alkoxy;

R₁₃, in each occurrence, is independently selected from among hydrogen, C₁₋₆ alkyls, C₂₋₆ alkenyl, C₂₋₆ alkynyl, C₃₋₁₉ branched alkyl, and C₃₋₈ cycloalkyl;

R₁₂, in each occurrence, is independently selected from among hydrogen, hydroxyl, C₁₋₆ alkyls, C₂₋₆ alkenyl, C₂₋₆ alkynyl, C₃₋₁₉ branched alkyl, C₃₋₈ cycloalkyl and C₁₋₆ alkoxy;

each of (a) and (d) is independently zero, 1, 2, or 3, and preferably 0;

each of (a1) and (a2) is independently zero, 1, 2, or 3, and preferably 1;

each (b) is independently zero, 1, 2, or 3, and preferably 0;

each (c) is independently zero, 1, 2, or 3, and preferably 1;

each (e) is independently zero or one, and preferably 0;

each (g) is independently zero or one, and preferably 1; and

(m) is a positive integer from about 2 to about 32 (e.g., 2, 4, 6, 8, 16, 32),

provided that (a) and (g) are not simultaneously zero, that one or more of Z₁ contain an oligonucleotide, and that one or more of Z₁ contain a folic acid.

In one embodiment, one Z₁ contains an oligonucleotide and the remaining Z₁ contains a folic acid.

For purposes of the present invention, (m) refers to the number of polymer arms. Each polymer arm includes a linear polymer such as polyethylene glycol. Preferably, (m) equals to from about 2 to about 32. For example, (m) is 32 when R₁ has 32 linear polymer arms. When (m) is 2, bisPEG is employed in the polymeric compounds described herein. Thus, the polymeric compounds can preferably include up to 32 polymer arms, i.e., 4, 8, 16 or 32. In one embodiment, the polymeric compounds can include four to eight polymer arms, where (m) can be from 4 to 8 (e.g., 4, 6 or 8). Preferably, the polymeric portion includes four polymer arms, where (m) is 4.

For purposes of the present invention, L₁ is the same or different when (a) is equal to or greater than 2.

For purposes of the present invention, L₂ is the same or different when (d) is equal to or greater than 2.

For purposes of the present invention, L₄ is the same or different when (a1) or (a2) is equal to or greater than 2.

For purposes of the present invention, C(R₄)(R₅) is the same or different when (b) is equal to or greater than 2.

For purposes of the present invention, C(R₆)(R₇) is the same or different when (c) is equal to or greater than 2.

In one embodiment, the tumor cells are prostate or cervical cancer cells.

In another aspect, the present invention provides a method of inhibiting a gene expression in mammalian cells or tissues. The method includes administering an effective amount of the compound of Formula (I) or a pharmaceutically acceptable salt thereof to a mammal in need thereof.

In one embodiment, the methods described herein are carried out using a compound of Formula (I′):

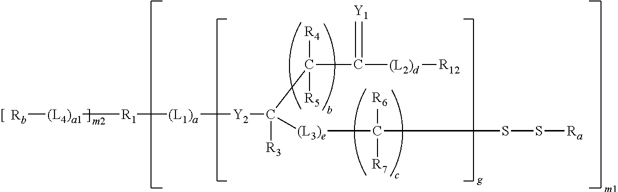

wherein

(m1) is a positive integer from about 1 to about 8 (e.g., 1, 2, 3, 4, 5, 6, 7, 8);

(m2) is zero or a positive integer from about 1 to about 7 (e.g. 0, 1, 2, 3, 4, 5, 6, 7); and

the sum of (m1) and (m2) is an integer from about 2 to about 8 (e.g., 2, 4, 6, 8).

In one particular embodiment, all (Z₁) contain an oligonucleotide. In this aspect, (m2) is zero and (m1) is 4 or 8. Alternatively, all (Z₁) are the same or different

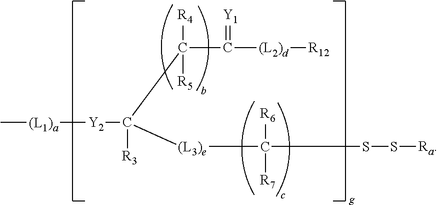

In another particular embodiment, one or more of Z₁ contain a folic acid. Alternatively, one or more Z₁ are -(L₄)ₐ₁-R_{b}. In this aspect, one Z₁ includes an oligonucleotide and each of the remaining Z₁ includes a folic acid, when (m) is greater than 2.

In a further embodiment, the compounds described herein include an optional diagnostic agent.

In another embodiment, each R₁₂ is the same or different groups selected from among H, NH₂, OH, CO₂H, C₁₋₆ alkoxy, C₁₋₆ alkyl, and preferably OH.

In yet another embodiment, each of R₃₋₇ is the same or different group selected from among hydrogen, methyl, ethyl and isopropyl. Preferably, R₃₋₇ are all hydrogen

In one preferred embodiment, (b), (d) and (e) are zero and (c) is 1.

In one preferred embodiment, the compounds of Formula (I) employed in the method described herein include Z₁ having the formula:

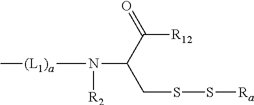

wherein,

(a) is 0 or 1;

(m) is 1, 2, 4, 8, 16 or 32;

R₁₂, in each occurrence, is independently selected from among hydroxyl, C₁₋₆ alkyls, C₂₋₆ alkenyl, C₂₋₆ alkynyl, C₃₋₁₉ branched alkyl, and C₁₋₆ alkoxy; and

all other variables are the same as defined above.

Alternatively, the compounds described herein have the formula:

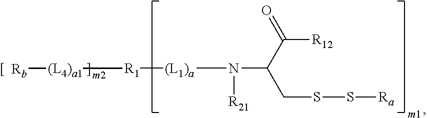

wherein (a) is 0 or 1.

In this respect, (m₂) is zero and all of Z₁ are

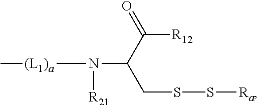

or

(m₁) is 1, or one Z₁ is

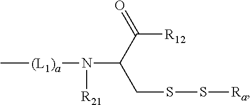

wherein each of remaining Z₁ includes a folic acid.

In a further embodiment, one Z₁ includes a diagnostic agent.

In another aspect, R₁ includes a polyalkylene oxide. Preferably, R₁ has the total number average molecular weight of from about 5,000 to about 25,000 daltons or from about 20,000 to about 45,000 daltons.

In one preferred aspect of the present invention, the methods described herein are conducted with the compounds having the formula:

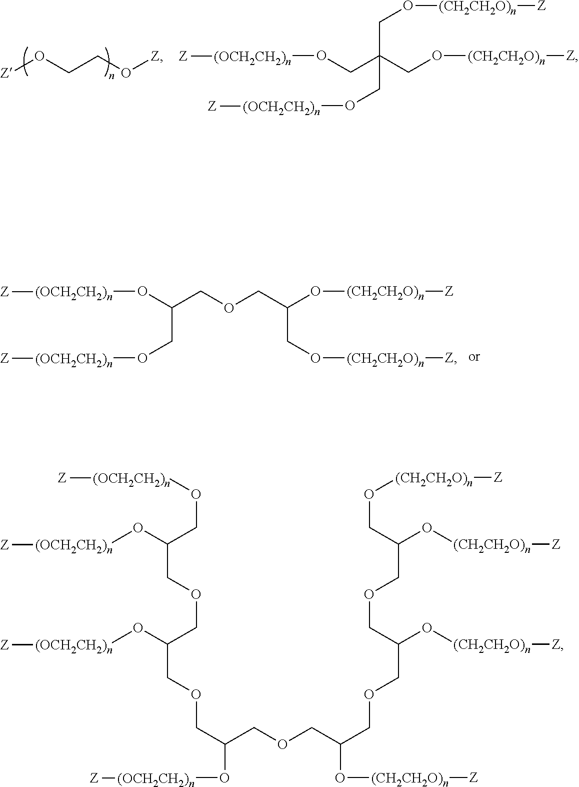

wherein
each Z is independently

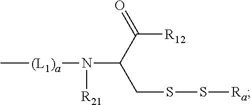

-(L₄)ₐ₁-R_{b}; and

-(L₄)ₐ₂-R_{c},

wherein

(a) is 0 or 1.

R₁₂, in each occurrence, is independently selected from among hydroxyl, C₁₋₆ alkyls, C₂₋₆ alkenyl, C₂₋₆ alkynyl, C₃₋₁₉ branched alkyl, and C₁₋₆ alkoxy;

(n) is a positive integer and the polymeric portion of the compound has the total number average molecular weight of from about 5,000 to about 25,000 daltons or from about 20,000 to about 45,000 daltons; and

all other variables are the same as defined above.

In this respect, all Z groups include an oligonucleotide. Alternatively, one Z includes an oligonucleotide and remaining one or more Z groups (e.g., 1, 2, 3, 4, 5, 6 or 7 Z groups) include a targeting agent such as folic acid. In a further embodiment, one Z includes an oligonucleotide, another Z includes a diagnostic agent, and remaining one or more (e.g., 2, 3, 4, 5, 6) Z include a folic acid.

In another aspect of the present invention, there are provided compounds of Formula (Ia):

R₁—{Z₁}ₘ

or a pharmaceutically acceptable salt thereof,

wherein

R₁ is a substantially non-antigenic water-soluble polymer;

each Z₁ is the same or different and selected from among

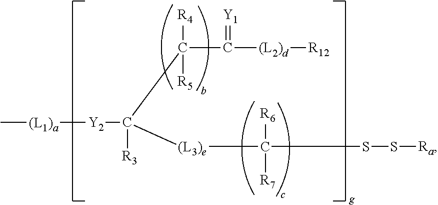

-(L₄)ₐ₁-R_{b}; and

-(L₄)ₐ₂-R_{c},

Y₁, in each occurrence, is independently S or O;

Y₂, in each occurrence, is independently NR₁₃;

Rₐ, in each occurrence, is the same or a different oligonucleotide;

each of L₁₋₄, in each occurrence, is the same or a different bifunctional linker;

R_{b}, in each occurrence, is a folic acid;

R_{c}, in each occurrence, is the same or a different diagnostic agent;

each of R₃₋₇ is independently selected from among hydrogen, C₁₋₆ alkyls, C₂₋₆ alkenyl, C₂₋₆ alkynyl, C₃₋₁₉ branched alkyl, C₃₋₈ cycloalkyl, and C₁₋₆ alkoxy;

R₁₃, in each occurrence, is independently selected from among hydrogen, C₁₋₆ alkyls, C₂₋₆ alkenyl, C₂₋₆ alkynyl, C₃₋₁₉ branched alkyl, and C₃₋₈ cycloalkyl;

R₁₂, in each occurrence, is independently selected from among hydrogen, hydroxyl, C₁₋₆ alkyls, C₂₋₆ alkenyl, C₂₋₆ alkynyl, C₃₋₁₉ branched alkyl, C₃₋₈ cycloalkyl and C₁₋₆ alkoxy;

each of (a) and (d) is independently zero, 1, 2, or 3;

each of (a1) and (a2) is independently zero, 1, 2, or 3;

each (b) is independently zero, 1, 2, or 3;

each (c) is independently zero, 1, 2, or 3;

each (e) is independently zero or one;

each (g) is independently zero or one; and

(m) is a positive integer from about 2 to about 32,

provided that (a) and (g) are not simultaneously zero, that one or more of Z₁ contain an oligonucleotide, and that one or more of Z₁ contain a folic acid.

In certain embodiments, variables are the same as defined in Formula (I).

### B. Substantially Non-Antigenic Water-Soluble Polymers

Polymers employed in the compounds described herein are preferably water soluble polymers and substantially non-antigenic such as polyalkylene oxides (PAO's).

In one aspect of the invention, the compounds described herein include a linear, terminally branched or multi-armed polyalkylene oxide. In some embodiments of the invention, the polyalkylene oxide includes polyethylene glycol and polypropylene glycol.

The polyalkylene oxide has the total number average molecular weight of from about 2,000 to about 100,000 daltons, preferably from about 5,000 to about 60,000 daltons. The polyalkylene oxide can be more preferably from about 5,000 to about 25,000 or from about 20,000 to about 45,000 daltons. In some particular embodiments, the compounds described herein include the polyalkylene oxide having the total number average molecular weight of from about 30,000 to about 45,000 daltons. In one particular embodiment, the polymeric portion has the total number average molecular weight of about 40,000 daltons.

The polyalkylene oxide includes polyethylene glycols and polypropylene glycols. More preferably, the polyalkylene oxide includes polyethylene glycol (PEG). PEG is generally represented by the structure:

—O—(CH₂CH₂O)ₙ—

where (n) represents the degree of polymerization for the polymer, and is dependent on the molecular weight of the polymer. Alternatively, the polyethylene glycol (PEG) residue portion of the compounds described herein can be selected from among:

—Y₇₁—(CH₂CH₂O)_{N}—CH₂CH₂Y₇₁—,

—Y_{71 —(CH}₂CH₂O)ₙ—CH₂C(═Y₇₂)—Y₇₁—,

—Y₇₁—(CH₂CH₂O)ₙ—CH₂C(═Y₇₂)Y₇₁—C(═Y₇₂)—,

—Y₇₁—C(═Y₇₂)—(CH₂)ₐ₇₁—Y₇₃—(CH₂CH₂O)ₙ—CH₂CH₂—Y₇₃—(CH₂)ₐ₇₁—C(═Y₇₂)—Y₇₁—, and

—Y₇₁—(CR₇₁R₇₂)ₐ₇₂—Y₇₃—(CH₂)_{b71}—O—(CH₂CH₂O)ₙ—(CH₂)_{b71}—Y₇₃—(CR₇₁R₇₂)ₐ₇₂—Y₇₁—,

wherein:

Y₇₁ and Y₇₃ are independently O, S, SO, SO₂, NR₇₃ or a bond;

Y₇₂ is O, S, or NR₇₄, preferably O;

R₇₁, R₇₂, R₇₃ and R₇₄ are independently selected from the same moieties which can be used for R₃;

(a71), (a72), and (b71) are independently zero or a positive integer (e.g. 0, 1, 2, 3), and preferably 1; and

(n) is an integer from about 10 to about 2300.

In one preferred aspect, the polymers useful in the compounds described herein include multi-arm PEG-OH or “star-PEG” products such as those described in NOF Corp. Drug Delivery System catalog, Ver. 8, April 2006, the disclosure of which is incorporated herein by reference. The polymers can be converted into suitably activated forms, using the activation techniques described in U.S. Pat. No. 5,122,614 or 5,808,096. Specifically, such a PEG can be of the formula:

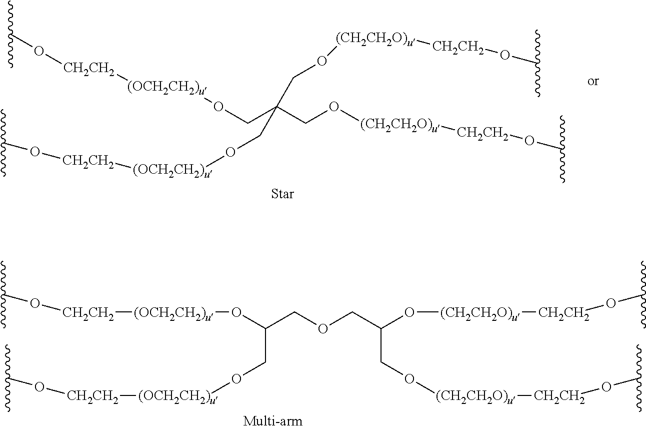

wherein:

(u′) is an integer from about 4 to about 455.

In one preferred embodiment, the degree of polymerization for the polymer (u′) is from about 28 to about 341 to provide polymers having the total number average molecular weight of from about 5,000 Da to about 60,000 Da, and preferably from about 114 to about 239 to provide polymers having the total number average molecular weight of from about 20,000 Da to about 42,000 Da. (u′) represents the number of repeating units in the polymer chain and is dependent on the molecular weight of the polymer. In one particular embodiment of the invention, (u′) is about 227 to provide the polymeric portion having the total number average molecular weight of about 40,000 Da.

In some preferred embodiments, all 4 of the PEG arms are converted to suitable activating groups, for facilitating attachment to oligonucleotides or folic acids. Such compounds prior to conversion include:

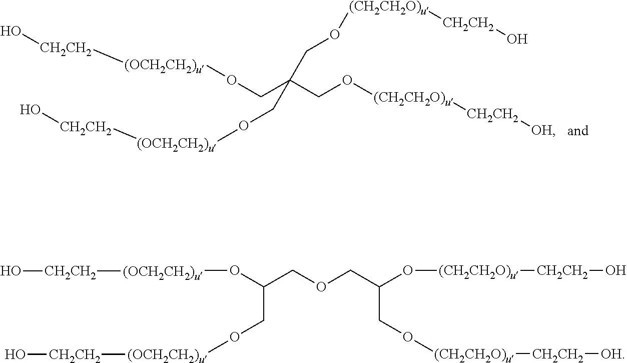

The polymeric substances included herein are preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

For purposes of the present invention, “substantially or effectively non-antigenic” means all materials understood in the art as being nontoxic and not eliciting an appreciable immunogenic response in mammals.

In some aspects, polymers having terminal carboxylic acid groups can be employed in the polymeric delivery systems described herein. Methods of preparing polymers having terminal carboxylic acids in high purity are described in U.S. Patent Application Publication No. 2007/0173615, the contents of which are incorporated herein by reference. The methods include first preparing a tertiary alkyl ester of a polyalkylene oxide followed by conversion to the carboxylic acid derivative thereof. The first step of the preparation of the PAO carboxylic acids of the process includes forming an intermediate such as t-butyl ester of polyalkylene oxide carboxylic acid. This intermediate is formed by reacting a PAO with a t-butyl haloacetate in the presence of a base such as potassium t-butoxide. Once the t-butyl ester intermediate has been formed, the carboxylic acid derivative of the polyalkylene oxide can be readily provided in purities exceeding 92%, preferably exceeding 97%, more preferably exceeding 99% and most preferably exceeding 99.5% purity.

In alternative aspects, polymers having terminal amine groups can be employed to make the compounds described herein. The methods of preparing polymers containing terminal amines in high purity are described in U.S. Patent Application Publication Nos. 2008/0249260 and 2007/0078219, the contents of each of which are incorporated by reference. For example, polymers having azides react with a phosphine-based reducing agent such as triphenylphosphine or an alkali metal borohydride reducing agent such as NaBH₄. Alternatively, polymers including leaving groups react with protected amine salts such as potassium salt of methyl-tert-butyl imidodicarbonate (KNMeBoc) or the potassium salt of di-tert-butyl imidodicarbonate (KNBoc₂) followed by deprotecting the protected amine group. The purity of the polymers containing the terminal amines formed by these processes is greater than about 95% and preferably greater than 99%.

### C. Bifunctional Linkers

Bifunctional linkers include amino acids, amino acid derivatives, and peptides. The amino acids can be among naturally occurring and non-naturally occurring amino acids. Derivatives and analogs of the naturally occurring amino acids, as well as various art-known non-naturally occurring amino acids (D or L), hydrophobic or non-hydrophobic, are also contemplated to be within the scope of the invention. A suitable non-limiting list of the non-naturally occurring amino acids includes 2-aminoadipic acid, 3-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4-aminobutyric acid, desmosine, 2,2-diaminopimelic acid, 2,3-diaminopropionic acid, N-ethylglycine, N-ethylasparagine, 3-hydroxyproline, 4-hydroxyproline, isodesmosine, alloisoleucine, N-methylglycine, sarcosine, N-methyl-isoleucine, 6-N-methyllysine, N-methylvaline, norvaline, norleucine, and ornithine. Some amino acid residues are selected from glycine, alanine, methionine or sarcosine, and more preferably, glycine.

In an alternative aspect of the present invention, L₁₋₄ are the same or different groups selected from among:

—[C(═O)]ᵥ(CR₂₂R₂₃)ₜ[C(═O)]_{v′}—,

—[C(═O)]ᵥ(CR₂₂R₂₃)ₜ—O[C(═O)]_{v′}—,

—[C(═O)]ᵥ(CR₂₂R₂₃)ₜ—NR₂₆[C(═O)]_{v′}—,

—[C(═O)]ᵥO(CR₂₂R₂₃)ₜ[C(═O)]_{v′}—,

—[C(═O)]ᵥO(CR₂₂R₂₃)ₜO[C(═O)]_{v′}—,

—[C(═O)]ᵥO(CR₂₂R₂₃)ₜNR₂₆[C(═O)]_{v′}—,

—[C(═O)]ᵥNR₂₁(CR₂₂R₂₃)ₜ[C(═O)]_{v′}—,

—[C(═O)]ᵥNR₂₁(CR₂₂R₂₃)ₜO[C(═O)]_{v′}—,

—[C(═O)]ᵥNR₂₁(CR₂₂R₂₃)ₜNR₂₆[C(═O)]_{v′}—,

—[C(═O)]ᵥ(CR₂₂R₂₃)ₜO—(CR₂₈R₂₉)_{t′}[C(═O)]_{v′}—,

—[C(═O)]ᵥ(CR₂₂R₂₃)ₜNR₂₆—(CR₂₈R₂₉)_{t′}[C(═O)]_{v′}—,

—[C(═O)]ᵥ(CR₂₂R₂₃)ₜS—(CR₂₈R₂₉)_{t′}[C(═O)]_{v′}—,

—[C(═O)]ᵥO(CR₂₂R₂₃)ₜO—(CR₂₈R₂₉)_{t′}[C(═O)]_{v′}—,

—[C(═O)]ᵥO(CR₂₂R₂₃)ₜNR₂₆—(CR₂₈R₂₉)_{t′}[C(═O)]_{v′}—,

—[C(═O)]ᵥO(CR₂₂R₂₃)ₜS—(CR₂₈R₂₉)_{t′}[C(═O)]_{v′}—,

—[C(═O)]ᵥNR₂₁(CR₂₂R₂₃)ₜO—(CR₂₈R₂₉)_{t′}[C(═O)]_{v′}—,

—[C(═O)]ᵥNR₂₁(CR₂₂R₂₃)ₜNR₂₆—(CR₂₈R₂₉)_{t′}[C(═O)]_{v′}—,

—[C(═O)]ᵥNR₂₁(CR₂₂R₂₃)ₜS—(CR₂₈R₂₉)_{t′}[C(═O)]_{v′}—,

—[C(═O)]ᵥ(CR₂₂R₂₃CR₂₈R₂₉O)ₜNR₂₆[C(═O)]_{v′}—,

—[C(═O)]ᵥ(CR₂₂R₂₃CR₂₈R₂₉O)ₜ[C(═O)]_{v′}—,

—[C(═O)]ᵥO(CR₂₂R₂₃CR₂₈R₂₉O)ₜNR₂₆[C(═O)]_{v′}—,

—[C(═O)]ᵥO(CR₂₂R₂₃CR₂₈R₂₉O)ₜ[C(═O)]_{v′}—,

—[C(═O)]ᵥNR₂₁(CR₂₂R₂₃CR₂₈R₂₉O)ₜNR₂₆[C(═O)]_{v′}—,

—[C(═O)]ᵥNR₂₁(CR₂₂R₂₃CR₂₈R₂₉O)ₜ[C(═O)]_{v′}—,

—[C(═O)]ᵥ(CR₂₂R₂₃CR₂₈R₂₉O)ₜ(CR₂₄R₂₅)_{t′}[C(═O)]_{v′}—,

—[C(═O)]ᵥO(CR₂₂R₂₃CR₂₈R₂₉O)ₜ(CR₂₄R₂₅)_{t′}[C(═O)]_{v′}—,

—[C(═O)]ᵥNR₂₁(CR₂₂R₂₃CR₂₈R₂₉O)ₜ(CR₂₄R₂₅)_{t′}[C(═O)]_{v′}—,

—[C(═O)]ᵥ(CR₂₂R₂₃CR₂₈R₂₉O)ₜ(CR₂₄R₂₅)_{t′}O[C(═O)]_{v′}—,

—[C(═O)]ᵥ(CR₂₂R₂₃)ₜ(CR₂₄R₂₅CR₂₈R₂₉O)_{t′}[C(═O)]_{v′}—,

—[C(═O)]ᵥ(CR₂₂R₂₃)ₜ(CR₂₄R₂₅CR₂₈R₂₉O)_{t′}NR₂₆[C(═O)]_{v′}—,

—[C(═O)]ᵥO(CR₂₂R₂₃CR₂₈R₂₉O)ₜ(CR₂₄R₂₅)_{t′}O[C(═O)]_{v′}—,

—[C(═O)]ᵥO(CR₂₂R₂₃)ₜ(CR₂₄R₂₅CR₂₈R₂₉O)_{t′}[C(═O)]_{v′}—,

—[C(═O)]ᵥO(CR₂₂R₂₃)ₜ(CR₂₄R₂₅CR₂₈R₂₉O)_{t′}NR₂₆[C(═O)]_{v′}—,

—[C(═O)]ᵥNR₂₁(CR₂₂R₂₃CR₂₈R₂₉O)ₜ(CR₂₄R₂₅)_{t′}O[C(═O)]_{v′}—,

—[C(═O)]ᵥNR₂₁(CR₂₂R₂₃)ₜ(CR₂₄R₂₅CR₂₈R₂₉O)_{t′}[C(═O)]_{v′}—,

—[C(═O)]ᵥNR₂₁(CR₂₂R₂₃)ₜ(CR₂₄R₂₅CR₂₈R₂₉O)_{t′}NR₂₆[C(═O)]_{v′}—,

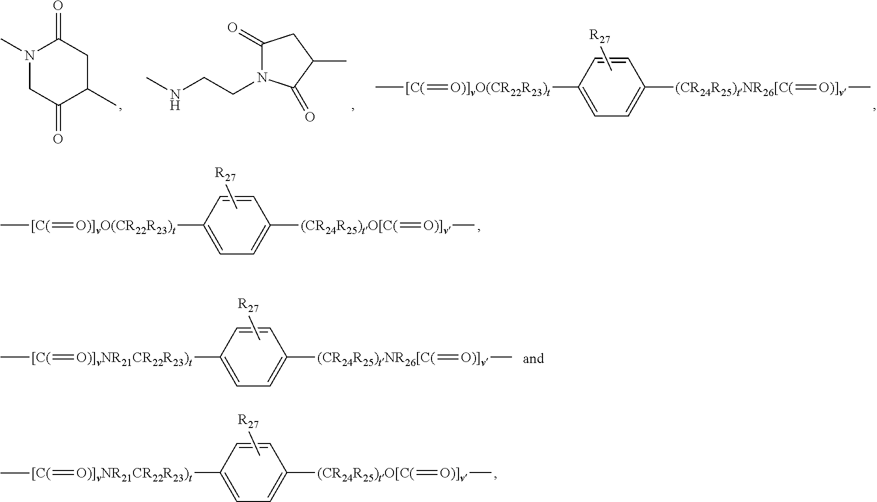

wherein:

R₂₁₋₂₉ are the same or different groups selected from among hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆ heteroalkyls, C₁₋₆ alkoxy, phenoxy and C₁₋₆heteroalkoxy;

(t) and (t′) are independently zero or a positive integer, preferably zero or an integer from about 1 to about 12, more preferably an integer from about 1 to about 8, and most preferably 1 or 2; and

(v) and (v′) are independently zero or 1.

In some preferred embodiments, L₁₋₄ of Formula (I) and Formula (Ia) (more preferably, L₄ of Formula (Ia)) include the same or different groups selected from among:

—[C(═O)]ᵣNH(CH₂)₂CH═N—NHC(═O)—(CH₂)₂—,

—[C(═O)]ᵣNH(CH₂)₂(CH₂CH₂O)₂(CH₂)₂NH[C(═O)]_{r′}—,

—[C(═O)]ᵣNH(CH₂CH₂)(CH₂CH₂O)₂NH[C(═O)]_{r′}—,

—[C(═O)]ᵣNH(CH₂CH₂)₂NH(CH₂CH₂)_{s′}[C(═O)]_{r′}—,

—[C(═O)]ᵣNH(CH₂CH₂)₂S(CH₂CH₂)_{s′}[C(═O)]_{r′}—,

—[C(═O)]ᵣNH(CH₂CH₂)(CH₂CH₂O)[C(═O)]_{r′}—,

—[C(═O)]ᵣNH(CH₂CH₂)₂O(CH₂CH₂)_{s′}[C(═O)]_{r′}—,

—[C(═O)]ᵣNH(CH₂CH₂O)(CH₂)NH[C(═O)]_{r′}—,

—[C(═O)]ᵣNH(CH₂CH₂O)₂(CH₂)[C(═O)]_{r′}—,

—[C(═O)]ᵣNH(CH₂CH₂O)ₛ(CH₂)_{s′}[C(═O)]_{r′}—,

—[C(═O)]ᵣNHCH₂CH₂NH[C(═O)]_{r′}—,

—[C(═O)]ᵣNH(CH₂CH₂)₂O[C(═O)]_{r′}—,

—[C(═O)]ᵣNH(CH₂CH₂O)[C(═O)]_{r′}—,

—[C(═O)]ᵣNH(CH₂CH₂O)₂[C(═O)]_{r′}—,

—[C(═O)]ᵣNH(CH₂)₃[C(═O)]_{r′}—,

—[C(═O)]ᵣO(CH₂CH₂O)₂(CH₂)[C(═O)]_{r′}—,

—[C(═O)]ᵣO(CH₂)₂NH(CH₂)₂[C(═O)]_{r′}—, —[C(═O)]ᵣO(CH₂CH₂O)₂NH[C(═O)]_{r′}—,

—[C(═O)]ᵣO(CH₂)₂O(CH₂)₂[C(═O)]_{r′}—,

—[C(═O)]ᵣO(CH₂)₂S(CH₂)₂[C(═O)]_{r′}—,

—[C(═O)]ᵣO(CH₂CH₂)NH[C(═O)]_{r′}—,

—[C(═O)]ᵣO(CH₂CH₂)O[C(═O)]_{r′}—,

—[C(═O)]ᵣO(CH₂)₃NH[C(═O)]_{r′}—,

—[C(═O)]ᵣO(CH₂)₃O[C(═O)]_{r′}—,

—[C(═O)]ᵣO(CH₂)₃[C(═O)]_{r′}—,

—[C(═O)]ᵣCH₂NHCH₂[C(═O)]_{r′}—,

—[C(═O)]ᵣCH₂OCH₂[C(═O)]_{r′}—,

—[C(═O)]ᵣCH₂SCH₂[C(═O)]_{r′}—,

—[C(═O)]ᵣS(CH₂)₃[C(═O)]_{r′}—,

—[C(═O)]ᵣ(CH₂)₃[C(═O)]_{r′}—,

—NH(CH₂CH₂O)₂(CH₂)₂NH[C(═O)]_{r′}—,

—NH(CH₂)₃—,

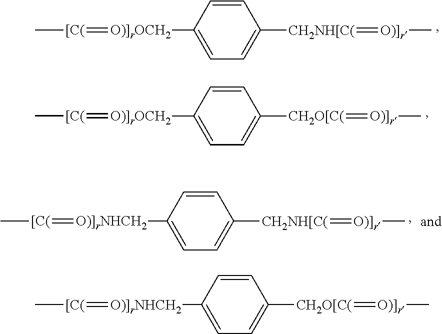

wherein (r) and (r′) are independently zero or 1; and (s) and (s′) are independently 1, 2, or 3. Both (r) and (r′) are not zero simultaneously.

For purposes of the present invention, when values for bifunctional linkers are positive integers equal to or greater than 2, the same or different bifunctional linkers can be employed. In one embodiment containing two or more bifunctional linkers, where (a), (a1), and (a2) are equal to or greater than 2, the bifunctional linkers can be the same or different.

### D. Diagnostic Agents

A further aspect of the invention provides the compounds optionally prepared with a diagnostic tag linked to the polymeric delivery system described herein, wherein the tag is selected for diagnostic or imaging purposes.

The compounds described herein can be labeled such as biotinylated compounds, fluorescent compounds (e.g., FAM), radiolabelled compounds. A suitable tag is prepared by linking any suitable moiety, e.g., an amino acid residue, to any art-standard emitting isotope, radio-opaque label, magnetic resonance label, or other non-radioactive isotopic labels suitable for magnetic resonance imaging, fluorescence-type labels, labels exhibiting visible colors and/or capable of fluorescing under ultraviolet, infrared or electrochemical stimulation, to allow for imaging tumor tissue during surgical procedures, and so forth. Optionally, the diagnostic tag is incorporated into and/or linked to a conjugated therapeutic moiety, allowing for monitoring of the distribution of a therapeutic biologically active material within an animal or human patient.

The tagged compounds are readily prepared, by art-known methods, with any suitable label, including, e.g., radioisotope labels. Simply by way of example, these include ¹³¹Iodine, ¹²⁵Iodine, ^{99m}Technetium and/or ¹¹¹Indium to produce radioimmunoscintigraphic agents for selective uptake into tumor cells, in vivo. For instance, there are a number of art-known methods of linking peptide to Tc-99m, including, simply by way of example, those shown by U.S. Pat. Nos. 5,328,679; 5,888,474; 5,997,844; and 5,997,845, incorporated by reference herein.

### E. Oligonucleotides

The compounds described herein can be used for delivering nucleic acids (oligonucleotides) into cells or tissues.

In order to more fully appreciate the scope of the present invention, the following terms are defined. The artisan will appreciate that the terms, “nucleic acid” or “nucleotide” apply to deoxyribonucleic acid (“DNA”), ribonucleic acid, (“RNA) whether single-stranded or double-stranded, unless otherwise specified, and any chemical modifications thereof. An “oligonucleotide” is generally a relatively short polynucleotide, e.g., ranging in size from about 2 to about 200 nucleotides, or more preferably from about 8 to about 30 nucleotides in length. The oligonucleotides according to the invention are generally synthetic nucleic acids, and are single stranded, unless otherwise specified. The terms, “polynucleotide” and “polynucleic acid” may also be used synonymously herein.

The oligonucleotides (analogs) are not limited to a single species of oligonucleotide but, instead, are designed to work with a wide variety of such moieties, it being understood that linkers can attach to one or more of the 3′- or 5′-terminals, usually PO₄ or SO₄ groups of a nucleotide. The nucleic acids molecules contemplated can include a phosphorothioate internucleotide linkage modification, sugar modification, nucleic acid base modification and/or phosphate backbone modification. The oligonucleotides can contain natural phosphorodiester backbone or phosphorothioate backbone or any other modified backbone analogs such as LNA (Locked Nucleic Acid), PNA (nucleic acid with peptide backbone), CpG oligomers, and the like, such as those disclosed at Tides 2002, Oligonucleotide and Peptide Technology Conferences, May 6-8, 2002, Las Vegas, Nev. and Oligonucleotide & Peptide Technologies, 18th & 19th November 2003, Hamburg, Germany, the contents of which are incorporated herein by reference.

Modifications to the oligonucleotides contemplated by the invention include, for example, the addition to or substitution of selected nucleotides with functional groups or moieties that permit covalent linkage of an oligonucleotide to a desirable polymer, and/or the addition or substitution of functional moieties that incorporate additional charge, polarizability, hydrogen bonding, electrostatic interaction, and functionality to an oligonucleotide. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodouracil, backbone modifications, methylations, base-pairing combinations such as the isobases isocytidine and isoguanidine, and analogous combinations. Oligonucleotides contemplated within the scope of the present invention can also include 3′ and/or 5′ cap structure.

For purposes of the present invention, “cap structure” shall be understood to mean chemical modifications, which have been incorporated at either terminus of the oligonucleotide. The cap can be present at the 5′-terminus (5′-cap) or at the 3′-terminus (3′-cap) or can be present on both terminus. A non-limiting examples of the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety. Details are described in WO 97/26270, incorporated by reference herein. The 3′-cap can includes, for example, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties. See also Beaucage and Iyer, 1993, Tetrahedron 49, 1925; the contents of which are incorporated by reference herein.

A non-limiting list of nucleoside analogs has the structure:

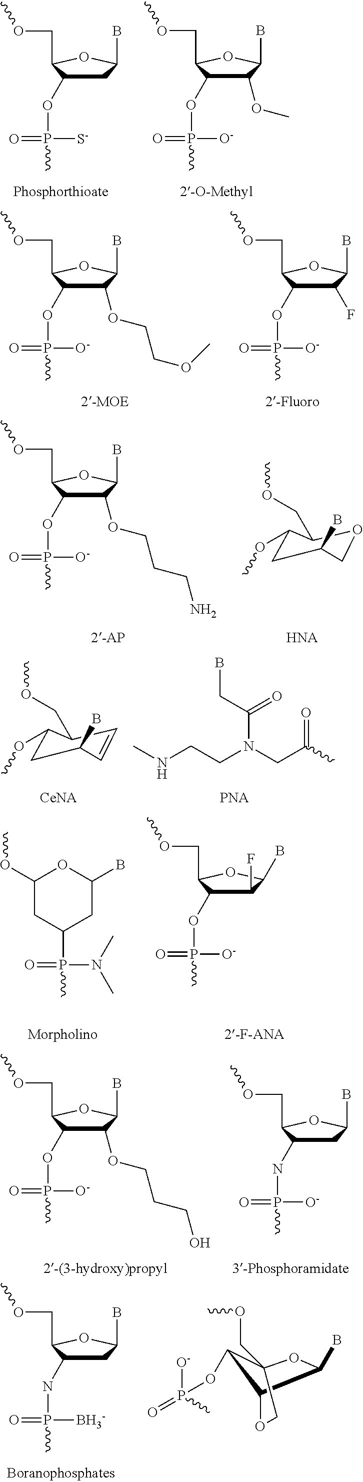

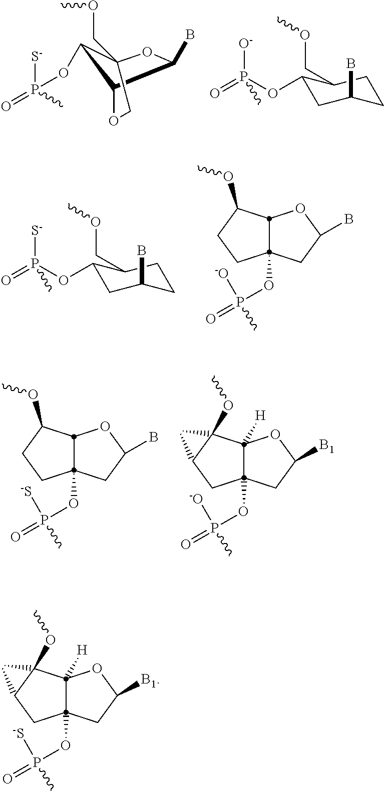

See more examples of nucleoside analogs described in Freier & Altmann; Nucl. Acid Res., 1997, 25, 4429-4443 and Uhlmann; Curr. Opinion in Drug Development, 2000, 3 (2), 293-213, the contents of each of which are incorporated herein by reference.

The term “antisense,” as used herein, refers to nucleotide sequences which are complementary to a specific DNA or RNA sequence that encodes a gene product or that encodes a control sequence. The term “antisense strand” is used in reference to a nucleic acid strand that is complementary to the “sense” strand. In the normal operation of cellular metabolism, the sense strand of a DNA molecule is the strand that encodes polypeptides and/or other gene products. The sense strand serves as a template for synthesis of a messenger RNA (“mRNA”) transcript (an antisense strand) which, in turn, directs synthesis of any encoded gene product. Antisense nucleic acid molecules may be produced by any art-known methods, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. In this manner, mutant phenotypes may be generated. The designations “negative” or (−) are also art-known to refer to the antisense strand, and “positive” or (+) are also art-known to refer to the sense strand.

For purposes of the present invention, “complementary” shall be understood to mean that a nucleic acid sequence forms hydrogen bond(s) with another nucleic acid sequence. A percent complementarity indicates the percentage of contiguous residues in a nucleic acid molecule which can form hydrogen bonds, i.e., Watson-Crick base pairing, with a second nucleic acid sequence, i.e., 5, 6, 7, 8, 9, 10 out of 10 being 50%, 60%, 70%, 80%, 90%, and 100% complementary. “Perfectly complementary” means that all the contiguous residues of a nucleic acid sequence form hydrogen bonds with the same number of contiguous residues in a second nucleic acid sequence.

In one embodiment, the choice for conjugation is an oligonucleotide (or “polynucleotide”) and after conjugation, the target is referred to as a residue of an oligonucleotide. The oligonucleotides can be selected from among any of the known oligonucleotides and oligodeoxynucleotides with phosphorodiester backbones or phosphorothioate backbones.

The oligonucleotides or oligonucleotide derivatives useful in the compounds described herein can include from about 8 to about 1000 nucleic acids, and preferably relatively short polynucleotides, e.g., ranging in size preferably from about 8 to about 30 nucleotides in length (e.g., about 8, 9, 10, 11, 12, 13, 14, 15, 16, 17, 18, 19, 20, 21, 22, 23, 24, 25, 26, 27, 28, 29, or 30).

In one aspect of useful nucleic acids used in the method described herein, oligonucleotides and oligodeoxynucleotides with natural phosphorodiester backbone or phosphorothioate backbone or any other modified backbone analogues include:

LNA (Locked Nucleic Acid);

PNA (nucleic acid with peptide backbone);

short interfering RNA (siRNA);

microRNA (miRNA);

nucleic acid with peptide backbone (PNA);

phosphorodiamidate morpholino oligonucleotides (PMO);

tricyclo-DNA;

decoy ODN (double stranded oligonucleotide);

catalytic RNA sequence (RNAi);

ribozymes;

aptamers;

spiegelmers (L-conformational oligonucleotides);

CpG oligomers, and the like, such as those disclosed at:

Tides 2002, Oligonucleotide and Peptide Technology Conferences, May 6-8, 2002, Las Vegas, Nev. and Oligonucleotide & Peptide Technologies, 18th & 19 Nov. 2003, Hamburg, Germany, the contents of which are incorporated herein by reference.

In another aspect of the nucleic acids used in the method described herein, oligonucleotides can include any suitable art-known nucleotide analogs and derivatives, including those listed by Table 1, below:

TABLE 1
Representative Nucleotide Analogs And Derivatives

| | |
|---|---|
| 4-acetylcytidine | 5-methoxyaminomethyl-2-thiouridine |
| 5-(carboxyhydroxymethyl)uridine | beta, D-mannosylqueuosine |
| 2′-O-methylcytidine | 5-methoxycarbonylmethyl-2-thiouridine |
| 5-methoxycarbonylmethyluridine | 5-carboxymethylaminomethyl-2-thiouridine |
| 5-methoxyuridine | 5-carboxymethylaminomethyluridine |
| Dihydrouridine | 2-methylthio-N6-isopentenyladenosine |
| 2′-O-methylpseudouridine | N-[(9-beta-D-ribofuranosyl-2-methylthiopurine-6- |
| | yl)carbamoyl]threonine |
| D-galactosylqueuosine | N-[(9-beta-D-ribofuranosylpurine-6-yl)N- |
| | methylcarbamoyl]threonine |
| 2′-O-methylguanosine | uridine-5-oxyacetic acid-methylester |
| 2′-halo-adenosine | 2′-halo-cytidine |
| 2′-halo-guanosine | 2′-halo-thymine |
| 2′-halo-uridine | 2′-halo-methylcytidine |
| 2′-amino-adenosine | 2′-amino-cytidine |
| 2′-amino-guanosine | 2′-amino-thymine |
| 2′-amino-uridine | 2′-amino-methylcytidine |
| Inosine | uridine-5-oxyacetic acid |
| N6-isopentenyladenosine | Wybutoxosine |
| 1-methyladenosine | Pseudouridine |
| 1-methylpseudouridine | Queuosine |
| 1-methylguanosine | 2-thiocytidine |
| 1-methylinosine | 5-methyl-2-thiouridine |
| 2,2-dimethylguanosine | 2-thiouridine |
| 2-methyladenosine | 4-thiouridine |
| 2-methylguanosine | 5-methyluridine |
| 3-methylcytidine | N-[(9-beta-D-ribofuranosylpurine-6-yl)- |
| | carbamoyl]threonine |
| 5-methylcytidine | 2′-O-methyl-5-methyluridine |
| N6-methyladenosine | 2′-O-methyluridine |
| 7-methylguanosine | Wybutosine |
| 5-methylaminomethyluridine | 3-(3-amino-3-carboxy-propyl)uridine |
| Locked-adenosine | Locked-cytidine |
| Locked-guanosine | Locked-thymine |
| Locked-uridine | Locked-methylcytidine |

Preferably, the oligonucleotide is involved in targeted tumor cells or downregulating a protein implicated in the resistance of tumor cells to anticancer therapeutics. For example, any art-known cellular proteins such as BCL-2 for downregulation by antisense oligonucleotides, for cancer therapy, can be used for the present invention. See U.S. patent application Ser. No. 10/822,205 filed Apr. 9, 2004, the contents of which are incorporated by reference herein. A non-limiting list of therapeutic oligonucleotides includes antisense HIF-1α oligonucleotides, antisense ErbB3 oligonucleotides, antisense survivin oligonucleotides and β-catenine oligonucleotides.

Preferably, the oligonucleotides useful in the method described herein include phosphorothioate backbone and LNA.

In one embodiment, the oligonucleotide useful in the method described herein includes antisense bcl-2 oligonucleotides, antisense HIF-1α oligonucleotides, antisense survivin oligonucleotides, and antisense Erbβ3 oligonucleotides.

In one preferred embodiment, the oligonucleotide can be, for example, an oligonucleotide that has the same or substantially similar nucleotide sequence as does Genasense (a/k/a oblimersen sodium, produced by Genta Inc., Berkeley Heights, N.J.). Genasense is an 18-mer phosphorothioate antisense oligonucleotide, TCTCCCAGCGTGCGCCAT (SEQ ID NO: 4), that is complementary to the first six codons of the initiating sequence of the human bcl-2 mRNA (human bcl-2 mRNA is art-known, and is described, e.g., as SEQ ID NO: 19 in U.S. Pat. No. 6,414,134, incorporated by reference herein). The U.S. Food and Drug Administration (FDA) gave Genasense Orphan Drug status in August 2000. Preferred embodiments include:

Preferred embodiments contemplated include:

(i) antisense Survivin LNA oligonucleotide

(SEQ ID NO: 1)

^{m}Cₛ-Tₛ-^{m}Cₛ-Aₛ-aₛ-tₛ-cₛ-cₛ-aₛ-tₛ-gₛ-gₛ-^{m}Cₛ-Aₛ-Gₛ-c;

where the upper case letter represents LNA, the “s” represents a phosphorothioate backbone;

(ii) antisense Bcl2 siRNA:

| | |
|---|---|
| SENSE | |
| 5′-gcaugcggccucuguuugadTdT-3′ | (SEQ ID NO: 2) |
| ANTISENSE | |
| 3′-dTdTcguacgccggagacaaacu-5′ | (SEQ ID NO: 3) |

where dT represents DNA;

(iii) Genasense (phosphorothioate antisense oligonucleotide):

(SEQ ID NO: 4)
tₛ-cₛ-tₛ-cₛ-cₛ-cₛ-aₛ-gₛ-cₛ-gₛ-tₛ-gₛ-cₛ-gₛ-cₛ-cₛ-
cₛ-aₛ-t

where the lower case letter represents DNA and “s” represents phosphorothioate backbone;

(iv) antisense HIF1α LNA oligonucleotide

(SEQ ID NO: 5)
5′- TₛGₛGₛcₛaₛaₛgₛcₛaₛtₛcₛcₛTₛGₛTₛa -3′

where the upper case letter represents LNA and the “s” represents phosphorothioate backbone.

(v) antisense ErbB3 LNA oligonucleotide

(SEQ ID NO: 6)
5′- TₛAₛGₛcₛcₛtₛgₛtₛcₛaₛcₛtₛtₛCₛTₛCₛ -3′

where the upper case letter represents LNA and the “s” represents phosphorothioate backbone.

LNA includes 2′-O, 4′-C methylene bicyclonucleotide as shown below:

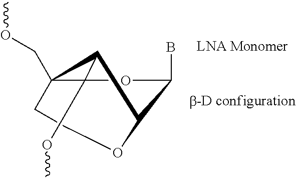

See detailed description of Survivin LNA disclosed in U.S. Patent Application Publication Serial Nos. 2006/0154888 and 2005/0014712, the contents of each of which is incorporated herein by reference. See detailed description of HIF-1α LNA disclosed in U.S. Patent Application Publication Nos. 2004/0096848, and 2006/0252721, the contents of each of which are incorporated herein by reference in its entirety. See also WO2008/113832, the contents of which are incorporated herein by reference in its entirety.

In one particular embodiment, the oligonucleotide comprises SEQ ID NO: 1, SEQ ID NOs 2 and 3, SEQ ID NO: 3, SEQ ID NO: 4, SEQ ID NO: 5, and SEQ ID NO: 6.

The oligonucleotides prior to the conjugation to the polymeric systems described herein include (CH₂)_{w} sulfhydryl linkers (thio oligonucleotides) at 5′ or 3′ end of the oligonucleotides, where (w) in this aspect is a positive integer of from about 1 to about 10 (1, 2, 3, 4, 5, 6, 7, 8, 9, 10, and preferably 4, 5, 6 or 7. The thio oligonucleotides have the structure SH—(CH₂)_{w}-Oligonucleotide. The compounds described herein can include oligonucleotides modified with hindered ester-containing (CH₂)_{w} sulfhydryl linkers. Prior to the attachment, the oligonucleotide has the structure:

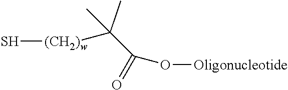

wherein (w) is a positive integer from about 1 to about 10, (e.g., 3, 4, 5, 6).

See WO 2008/034119, the contents of which are incorporated by reference. The polymeric compounds can release the oligonucleotides without thiol tail.

In one particular embodiment, 5′ end of the sense strand of siRNA is modified. For example, siRNA employed in the polymeric conjugates is modified with a 5′-C₆—SH. One particular embodiment of the present invention employs Bcl2-siRNA having the sequence of

| | |
|---|---|
| SENSE | 5′-SH-C₆-GCAUGCGGCCUCUGUUUGAdTdT-3′ |
| ANTISENSE | 3′-dTdTCGUACGCCGGAGACAAACU-5′. |

Examples of the modified oligonucleotides include:

(i) Genasense modified with a C₆—SH tail:

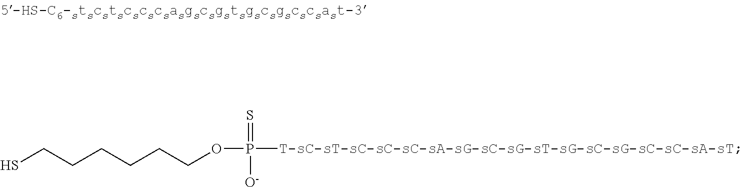

(ii) antisense HIF1α LNA modified with a C₆—SH tail:

5′- HS-C₆- ₛTₛGₛGₛcₛaₛaₛgₛcₛaₛtₛcₛcₛTₛGₛTₛa -3′;

(iii) antisense Survivin LNA modified with a C₆—SH tail

5′- HS- C₆- ₛ^{m}CₛTₛ^{m}CₛAₛaₛtₛcₛcₛaₛtₛgₛgₛ^{m}CₛAₛGₛc -3′;

(iv) antisense ErbB3 LNA modified with a C6-SH tail:

5′- HS- C₆- TₛAₛGₛcₛcₛtₛgₛtₛcₛaₛcₛtₛtₛCₛTₛCₛ -3′;

(v) Genasense modified with a hindered ester tail

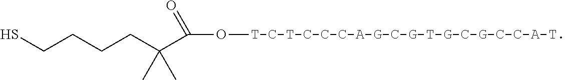

### F. Synthesis of the Polymeric Delivery Systems

Generally, the methods of preparing compounds described herein include reacting an activated polymer with an oligonucleotide modified with a SH group. Activated polymers useful in the methods described herein include a polymer containing a pyridyl disulfide group at the distal end. The methods provide a polymeric conjugate where the biologically active moiety is bonded to the polymer through —S—S— bond.

In one aspect of the invention, methods of preparing polymeric compounds described herein include:

reacting a polymeric compound of Formula (III):

R₁—{Z₁₁}ₘ₁₁

with an olignucleotide modified with a sulfhydryl group-containing moiety under conditions sufficient to form a compound of Formula (I):

R₁—{Z₁}ₘ

wherein

R₁ is a substantially non-antigenic water-soluble polymer;

each Z₁₁ is the same or different and selected from among

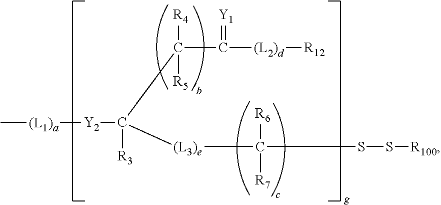

-(L₄)ₐ₁-R_{b} such as

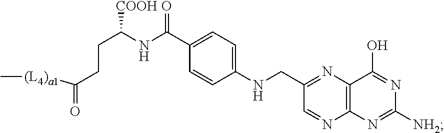

and
-(L₄)ₐ₂-R_{c},

Y₁, in each occurrence, is independently S or O, preferably O;

Y₂, in each occurrence, is independently NR₁₃, preferably NH;

R₁₀₀, in each occurrence is the same or different and selected from among H, a leaving group, an activating group, and

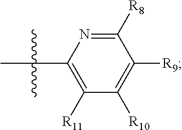

each of L₁₋₄, in each occurrence, is the same or a different bifunctional linker;

R_{b}, in each occurrence, is a folic acid;

R_{c}, in each occurrence, is the same or a different diagnostic agent;

each of R₃₋₇ is independently selected from among hydrogen, C₁₋₆ alkyls, C₂₋₆ alkenyl, C₂₋₆ alkynyl, C₃₋₁₉ branched alkyl, C₃₋₈ cycloalkyl, and C₁₋₆ alkoxy;

each of R₈₋₁₁ is an electron-withdrawing group such as substituted amido, acyl, azido, carboxy, alkyloxycarbonyl, cyano, and nitro, and preferably R₈ is nitro, and R₉, R₁₀ and R₁₁ are hydrogen;

R₁₃, in each occurrence, is independently selected from the group consisting of hydrogen, C₁₋₆ alkyls, C₂₋₆ alkenyl, C₂₋₆ alkynyl, C₃₋₁₉ branched alkyl, and C₃₋₈ cycloalkyl;

R₁₂, in each occurrence, is independently selected from the group consisting of hydrogen, hydroxyl, C₁₋₆ alkyls, C₂₋₆ alkenyl, C₂₋₆ alkynyl, C₃₋₁₉ branched alkyl, C₃₋₈ cycloalkyl and C₁₋₆ alkoxy;

each of (a) and (d) is independently zero, 1, 2, or 3, and preferably 0;

each of (a1) and (a2) is independently zero, 1, 2, or 3, and preferably 1;

each (b) is independently zero, 1, 2, or 3, and preferably 0;

each (c) is independently zero, 1, 2, or 3, and preferably 1;

each (e) is independently zero or one, and preferably 0;

each (g) is independently zero or one, and preferably 1; and

(m11) is a positive integer from about 2 to about 32 (e.g., 2, 4, 6, 8, 16, 32),

provided that (a) and (g) are not simultaneously zero and provided that one or more of Z₁₁ contain

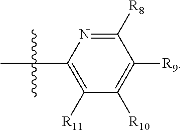

Preferably, the reactions are carried out in an inert solvent such as methylene chloride, chloroform, DMF or mixtures thereof. The reactions can be preferably conducted in the presence of a base, such as dimethylaminopyridine (DMAP), diisopropylethylamine, pyridine, triethylamine, etc. to neutralize any acids generated. The reactions can be carried out at a temperature from about 0° C. up to about 22° C. (room temperature). See detailed description in WO/2009/025669, the contents of which are incorporated herein by reference.

### G. Compounds of Formula (I)

Some particular embodiments prepared by the methods described herein have the structure:

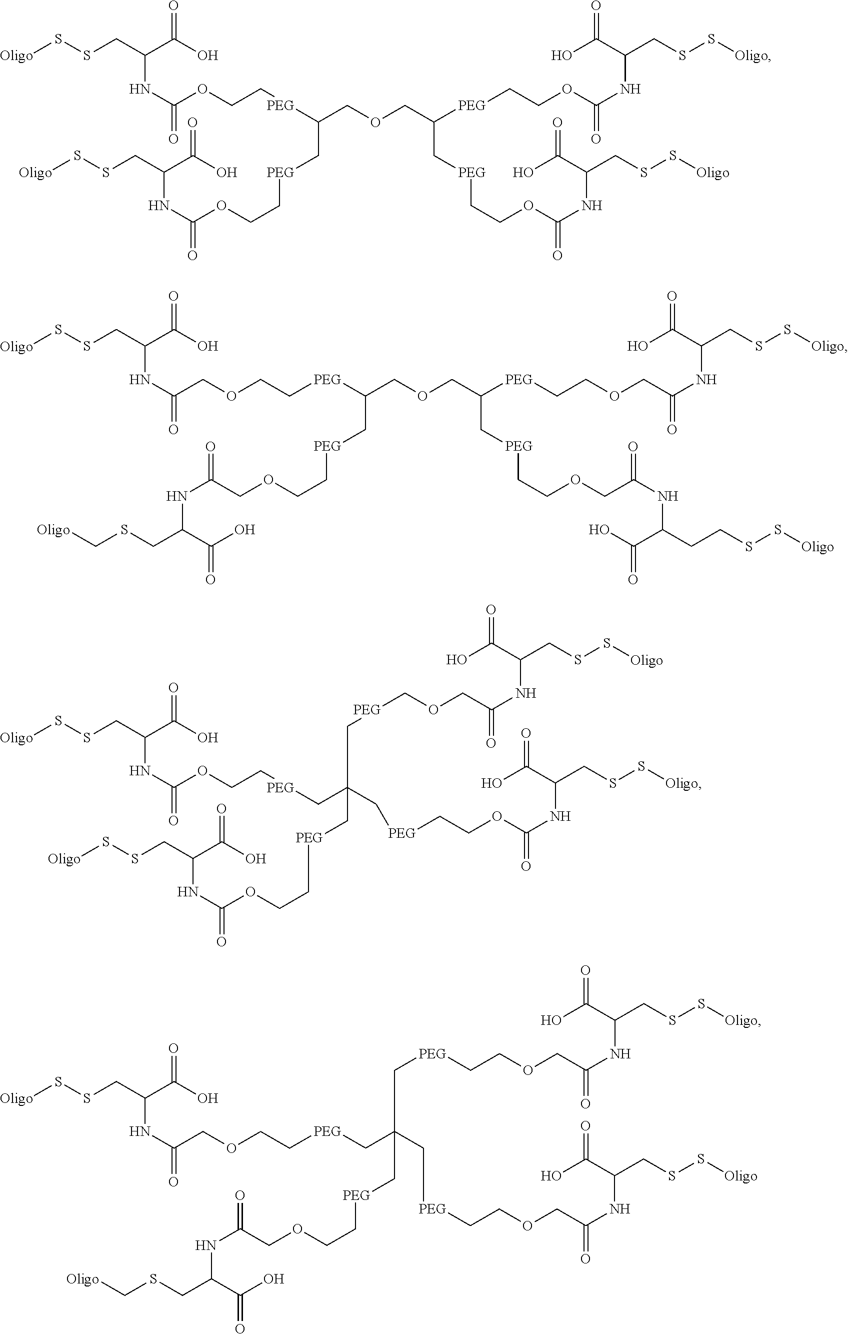

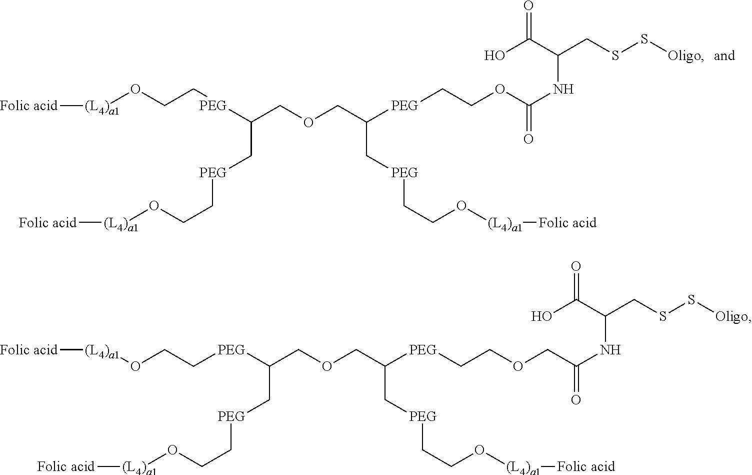

wherein:

Oligo is an oligonucleotide, preferably an oligonucleotide modified with a C₃-C₆ alkyl (C₆ alkyl);

PEG is a polyethylene glycol and the polymeric portion of the compound has the total number average molecular weight of from about 5,000 to about 25,000 daltons or from about 20,000 to about 45,000 daltons;

(a1) is one;

L₄ is —NH(CH₂CH₂O)₂(CH₂)₂NH[C(═O)]_{r′}— or —NH(CH₂)₃—, wherein (r′) is zero or one; and

all other variables are the same as defined above.

For example, compounds prepared by the method described herein include

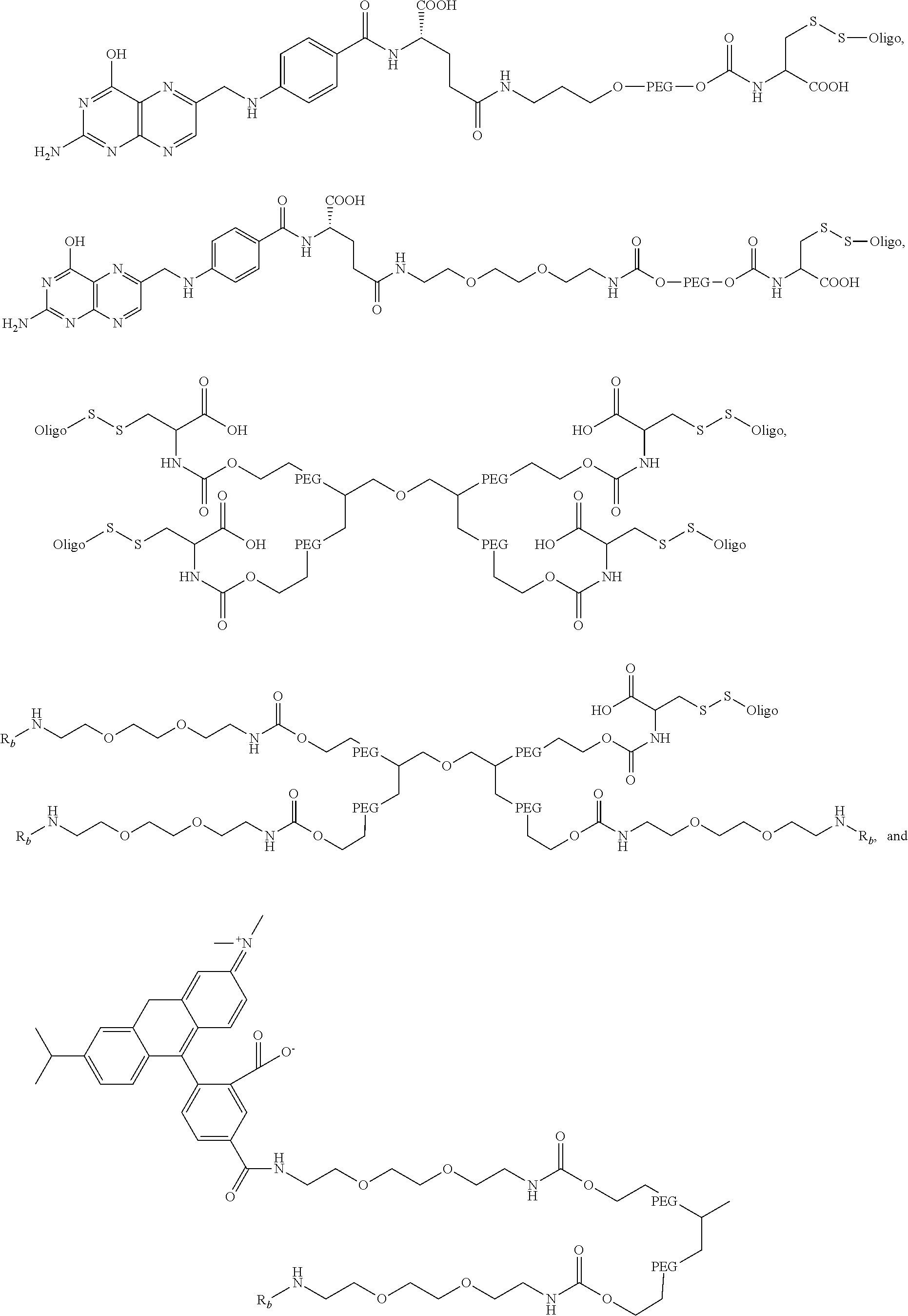

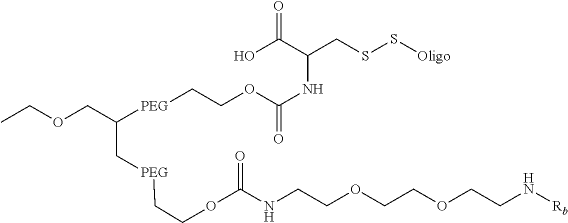

wherein

Oligo is an oligonucleotide;

R_{b} is

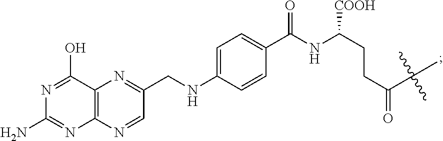

and

PEG is polyethylene glycol and the polymeric portion of the compound has the total number average molecular weight of from about 5,000 to about 25,000 daltons or from about 20,000 to about 45,000 daltons.

In a further embodiment, compounds prepared by the methods described herein include:

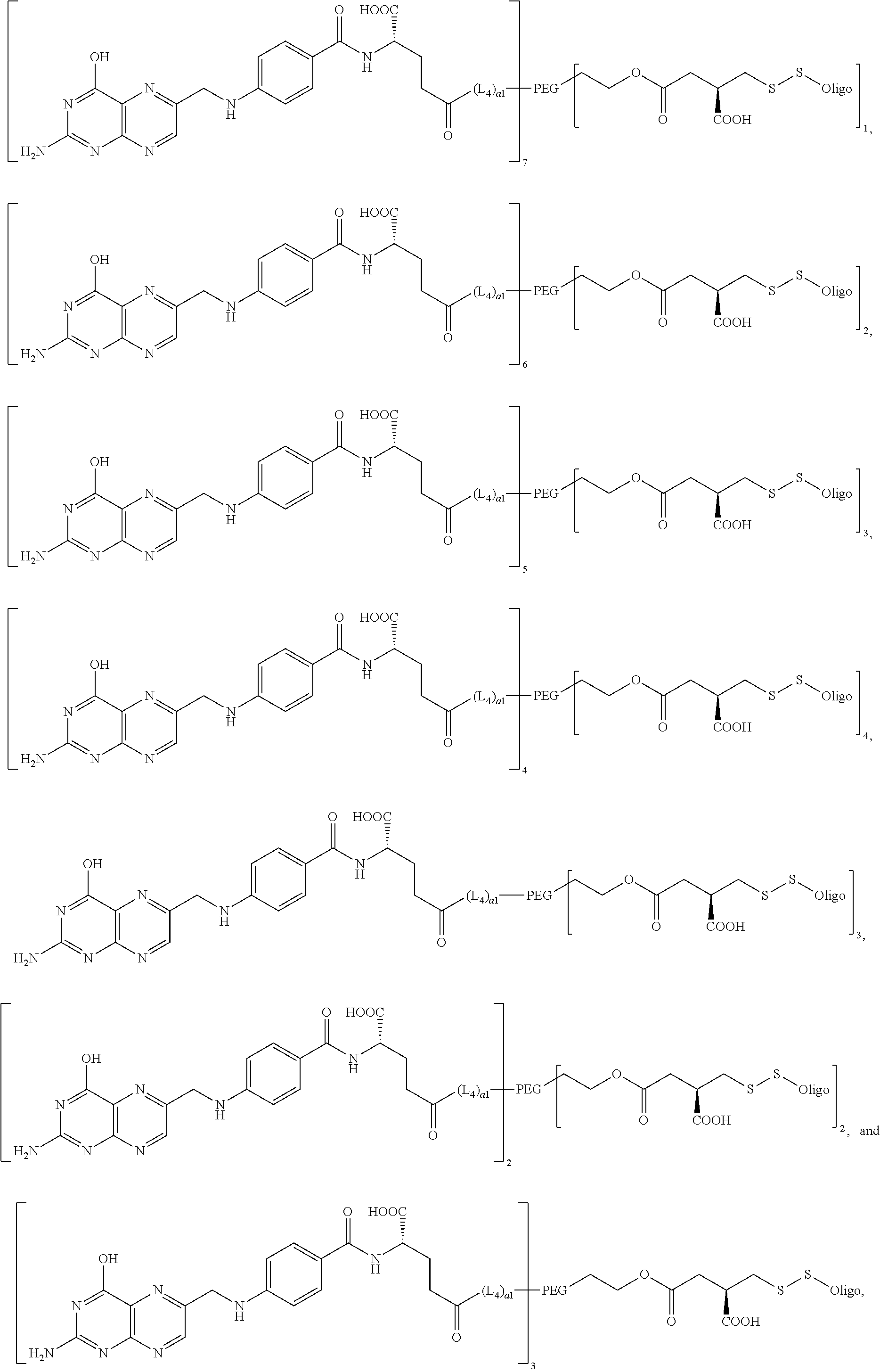

wherein

Oligo is an oligonucleotide, preferably an oligonucleotide modified with a C₃-C₆ alkyl (e.g., C₆ alkyl); and

PEG is a polyethylene glycol and the polymeric portion of the compound has the total number average molecular weight of from about 5,000 to about 25,000 daltons or from about 20,000 to about 45,000 daltons. For ease of the description and not limitation, the multi-arm PEG is shown as “PEG”. One arm, up to seven arms of the eight-arm PEG (or up to three arms of the four-arm PEG) can be conjugated with a folic acid.

Preferably, the compounds include therapeutic oligonucleotides such as antisense ErbB3 oligonucleotides and antisense Survivin oligonucleotides. For example, the compounds include a C₆-tail modified antisense LNA as follows:

5′-(CH₂)₆-TsAsGsCsCsTsGsTs CsAsCsTsTsCsTsCs-3′
or
5′-(CH₂)₆-GsCsTsGsCsCsAsTsGsGsAsTsTsGsAsG-3′,

wherein “s” represents a phosphorothioate linkage and the first three nucleotides in 5′ and 3′ terminal are LNA.

Preferably, the average molecular weight of the polymeric portion is about 40,000 daltons.

### H. Methods of Treatment

One aspect of the present invention provides methods of introducing or delivering an oligonucleotide into a mammalian cell. The method according to the present invention includes contacting a cell with a compound of Formula (I) described herein.

The present invention is useful for introducing oligonucleotides to a mammal having tumor cells. The compounds described herein can be administered to a mammal, preferably human.

According to the present invention, the present invention preferably provides methods of inhibiting or downregulating (or modulating) a gene expression in mammalian cells or tissues. The downregulation or inhibition of gene expression can be achieved in vivo and/or in vitro. The methods include contacting human cells or tissues with compounds of Formula (I) described herein. Once the contacting has occurred, successful inhibition or down-regulation of gene expression such as in mRNA or protein levels shall be deemed to occur when at least about 10%, preferably at least about 20% or higher is realized when measured in vivo or in vitro, when compared to that observed in the absence of the treatment with the compound described herein.

For purposes of the present invention, “inhibiting” or “down-regulating” shall be understood to mean that the expression of a target gene, or level of RNAs or equivalent RNAs encoding one or more protein subunits, or activity of one or more protein subunits, such as ErbB3, is reduced below that observed in the absence of the treatment with the conjugates described herein.

Preferably, gene expression of a target gene is inhibited in prostate or cervical cancer cells or tissues, for example, prostate or cervical cancer cells.

In a further embodiment, the cancer cells or tissues can be from one or more of the following: solid tumors, lymphomas, small cell lung cancer, acute lymphocytic leukemia (ALL), pancreatic cancer, glioblastoma, ovarian cancer, gastric cancer, breast cancer, colorectal cancer, ovarian cancer and brain tumors, etc.

In one particular embodiment, the compounds according to the methods described herein include, for example, antisense bcl-2 oligonucleotides, antisense HIF-1α oligonucleotides, antisense Survivin oligonucleotides, and antisense Erbβ3 oligonucleotides.

Preferably, the administering step is via the blood stream of the mammal.

A further aspect of the present invention provides methods of treatment for various medical conditions in mammals. The methods include administering, to the mammal in need of such treatment, an effective amount of a pharmaceutical composition containing a compound of Formula (I). The polymeric conjugate compounds are useful for, among other things, treating diseases including, but not limited to, cancer, inflammatory disease, and autoimmune disease.

In this aspect, a useful target gene includes, but is not limited to, oncogenes, pro-angiogenesis pathway genes, pro-cell proliferation pathway genes, viral infectious agent genes, and pro-inflammatory pathway genes.

In yet a further aspect, there are also provided methods of treating a patient having a malignancy or cancer, comprising administering an effective amount of a pharmaceutical composition containing the compound of Formula (I) to a patient in need thereof. In alternative aspects, the cancer being treated can be one or more of the following: solid tumors, lymphomas, small cell lung cancer, acute lymphocytic leukemia (ALL), pancreatic cancer, glioblastoma, ovarian cancer, gastric cancers, colorectal cancer, etc. The compositions are useful for treating neoplastic disease, reducing tumor burden, preventing metastasis of neoplasms and preventing recurrences of tumor/neoplastic growths in mammals by downregulating gene expression of a target gene.

Any oligonucleotide, etc. which has therapeutic effects in the unconjugated state can be used in its conjugated form, made as described herein.

In one particular embodiment, the methods described herein include administering polynucleotides (oligonucleotides), preferably antisense oligonucleotides to mammalian cells. The methods include delivering an effective amount of a conjugate prepared as described herein to the condition being treated will depend upon the polynucleotides efficacy for such conditions.

For example, if the unconjugated oligonucleotides (for example antisense ErbB3 oligonucleotides, antisense Survivin oligonucleotides) has efficacy against certain cancer or neoplastic cells, the method would include delivering a polymer conjugate containing the oligonucleotides to the cells having susceptibility to the native oligonucleotides. The delivery can be made in vivo as part of a suitable pharmaceutical composition or directly to the cells in an ex vivo environment. In one particular treatment, the polymeric conjugates including oligonucleotides (SEQ ID NO. 1, SEQ ID NOs: 2 and 3, SEQ ID NO: 4, SEQ ID NO: 5, SEQ ID NO: 6) can be used.

In yet another aspect, the present invention provides methods of inhibiting the growth or proliferation of cancer cells in vivo or in vitro. The methods include contacting cancer cells with the compounds described herein. Alternatively, the present invention provides methods of modulating apoptosis in cancer cells by administering the compounds described herein to a mammal in need thereof.

In yet another aspect, there are also provided methods of increasing the sensitivity of cancer cells or tissues to chemotherapeutic agents in vivo or in vitro. In one particular aspect, the methods include introducing the oligonucleotide (antisense LNA) conjugates described herein to cancer cells to reduce survivin expression in the cancer cells or tissues, wherein the antisense oligonucleotide binds to mRNA expressed from the survivin gene and reduces survivin gene expression.

In yet another aspect, there are provided methods of killing tumor cells in vivo or in vitro. The methods include introducing the compounds described herein to tumor cells to reduce gene expression such as ErbB3 and contacting the tumor cells with an amount of at least one chemotherapeutic agent sufficient to kill a portion of the tumor cells. Thus, the portion of tumor cells killed can be greater than the portion which would have been killed by the same amount of the chemotherapeutic agent in the absence of the compounds described herein.

In a further aspect of the invention, a chemotherapeutic agent can be used in combination, simultaneously or sequentially, with the methods employing the compounds described herein. The compounds described herein can be administered concurrently with the chemotherapeutic agent, prior to, or after the administration of the chemotherapeutic agent. Thus, the compounds described herein can be administered during or after treatment of the chemotherapeutic agent.

### I. Pharmaceutical Compositions/Formulations

Pharmaceutical compositions including the compounds described herein may be formulated in conjunction with one or more physiologically acceptable carriers comprising excipients and auxiliaries which facilitate processing of the active compounds into preparations which can be used pharmaceutically. Proper formulation is dependent upon the route of administration chosen, i.e., whether local or systemic treatment is treated. Parenteral routes are preferred in many aspects of the invention.

Administration of pharmaceutical compositions containing the compounds of Formula (I) described herein may be oral, pulmonary, topical including epidermal, transdermal, ophthalmic and to mucous membranes including vaginal and rectal delivery or parenteral including intravenous, intraarterial, subcutaneous, intraperitoneal or intramuscular injection or infusion. In one embodiment, the compounds containing therapeutic oligonucleotides is administered IV, IP or as a bolus injection.

For injection, including, without limitation, intravenous, intramuscular and subcutaneous injection, the compounds described herein may be formulated in aqueous solutions, preferably in physiologically compatible buffers such as physiological saline buffer or polar solvents including, without limitation, a pyrrolidone or dimethylsulfoxide.

The compounds may also be formulated for parenteral administration, e.g., by bolus injection or continuous infusion. Formulations for injection may be presented in unit dosage form, e.g., in ampoules or in multi-dose containers. Useful compositions include, without limitation, suspensions, solutions or emulsions in oily or aqueous vehicles, and may contain adjuncts such as suspending, stabilizing and/or dispersing agents. Pharmaceutical compositions for parenteral administration include aqueous solutions of a water soluble form, such as, without limitation, a salt (preferred) of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances that increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions. Alternatively, the active ingredient may be in powder form for constitution with a suitable vehicle, e.g., sterile, pyrogen-free water, before use.

For oral administration, the compounds can be formulated by combining the active compounds with pharmaceutically acceptable carriers well-known in the art. Such carriers enable the compounds of the invention to be formulated as tablets, pills, lozenges, dragees, capsules, liquids, gels, syrups, pastes, slurries, solutions, suspensions, concentrated solutions and suspensions for diluting in the drinking water of a patient, premixes for dilution in the feed of a patient, and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropyl methylcellulose, sodium carboxymethylcellulose, and/or polyvinylpyrrolidone (PVP). If desired, disintegrating agents may be added, such as cross-linked polyvinyl pyrrolidone, agar, or alginic acid. A salt such as sodium alginate may also be used.

For administration by inhalation, the compounds of the present invention can conveniently be delivered in the form of an aerosol spray using a pressurized pack or a nebulizer and a suitable propellant.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

Other delivery systems such as liposomes and emulsions can also be used.

Additionally, the conjugates may be delivered using a sustained-release system, such as semi-permeable matrices of solid hydrophobic polymers containing the therapeutic agent. Various sustained-release materials have been established and are well known by those skilled in the art.

### J. Dosages

Determination of a therapeutically effective amount is well within the capability of those skilled in the art, especially in light of the disclosure herein.

For any conjugate used in the methods of the invention, the therapeutically effective amount can be estimated initially from in vitro assays. Then, the dosage can be formulated for use in animal models so as to achieve a circulating concentration range that includes the effective dosage. Such information can then be used to more accurately determine dosages useful in patients.

The amount of the composition, e.g., used as a prodrug, that is administered will depend upon the parent molecule included therein (i.e., efficacy of an unconjugated oligonucleotide). Generally, the amount of prodrug used in the treatment methods is that amount which effectively achieves the desired therapeutic result in mammals. Naturally, the dosages of the various prodrug compounds will vary somewhat depending upon the parent compound (oligonucleotides such as LNA), rate of in vivo hydrolysis, molecular weight of the polymer, etc. In addition, the dosage, of course, can vary depending upon the dosage form and route of administration. In general, however, the oligonucleotide conjugates described herein can be administered in amounts ranging from about 1 mg/kg/week to about 1 g/kg/week, preferably from about 1 to about 500 mg/kg/week and more preferably from 1 to about 100 mg/kg/week (i.e., from about 2 to about 60 mg/kg/week). The range set forth above is illustrative and those skilled in the art will determine the optimal dosing of the prodrug selected based on clinical experience and the treatment indication. Moreover, the exact formulation, route of administration and dosage can be selected by the individual physician in view of the patient's condition. Additionally, toxicity and therapeutic efficacy of the compounds described herein can be determined by standard pharmaceutical procedures in cell cultures or experimental animals using methods well-known in the art.

Alternatively, dosage levels of the order of from about 0.1 mg to about 140 mg per kilogram of body weight per day are useful in the treatment of the above-indicated conditions (about 0.5 mg to about 7 g per subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

In one embodiment, the treatment of the present invention includes administering the oligonucleotide conjugates described herein in an amount of from about 2 to about 50 mg/kg/dose, such as from about 5 to about 30 mg/kg/dose to a mammal.

Alternatively, the delivery of the oligonucleotide conjugates described herein includes contacting a concentration of oligonucleotides of from about 0.1 to about 1000 nM, preferably from about 10 to about 1000 nM with tumor cells or tissues in vivo or in vitro.

The compositions may be administered once daily or divided into multiple doses which can be given as part of a multi-week treatment protocol. The precise dose will depend on the stage and severity of the condition, the susceptibility of the tumor to the polymer-prodrug composition, and the individual characteristics of the patient being treated, as will be appreciated by one of ordinary skill in the art.

In all aspects of the invention where polymeric conjugates are administered, the dosage amount mentioned is based on the amount of oligonucleotide molecule rather than the amount of polymeric conjugate administered. It is contemplated that the treatment will be given for one or more days until the desired clinical result is obtained. The exact amount, frequency and period of administration of the compound of the present invention will vary, of course, depending upon the sex, age and medical condition of the patent as well as the severity of the disease as determined by the attending clinician.

Still further aspects include combining the compound of the present invention described herein with other anticancer therapies for synergistic or additive benefit.

### EXAMPLES

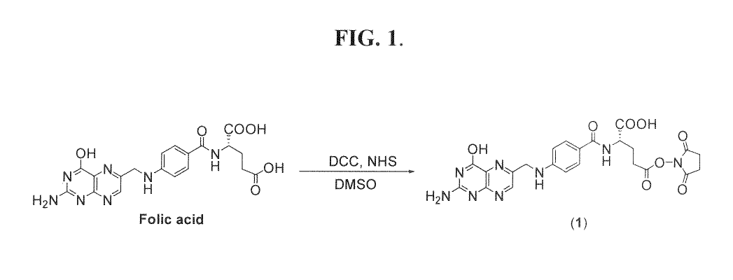

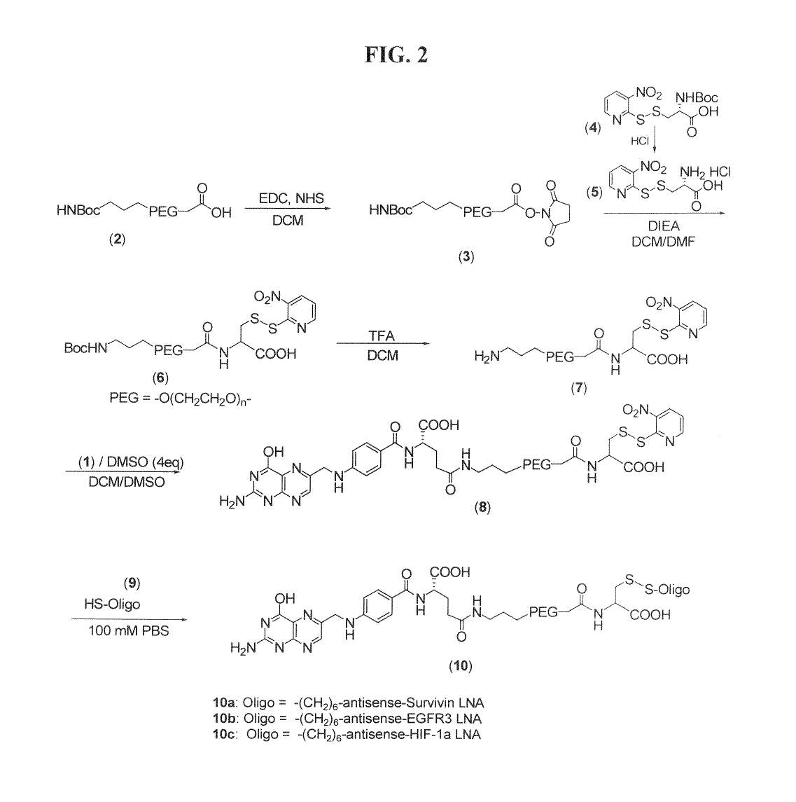

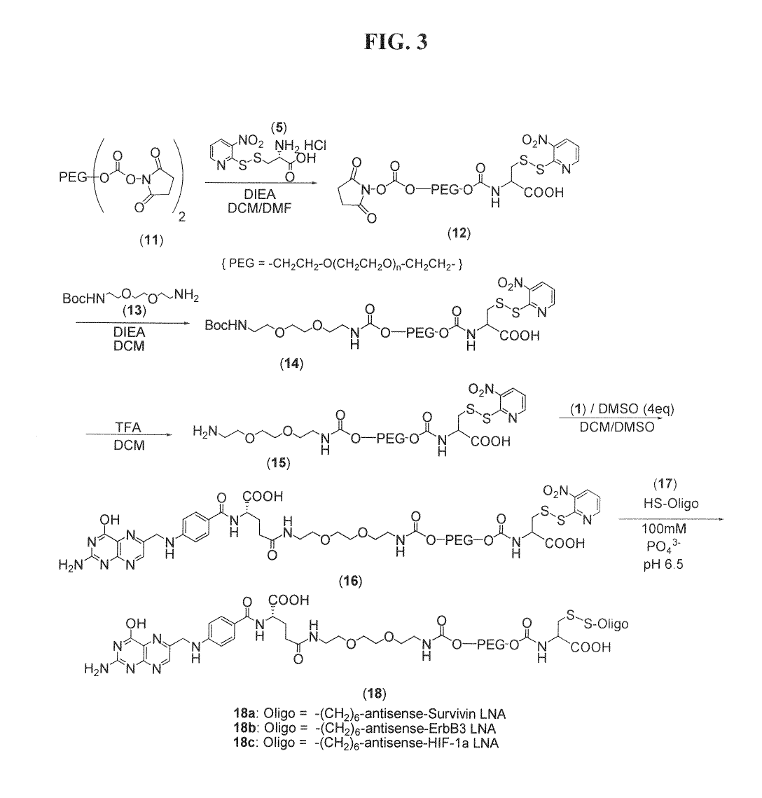

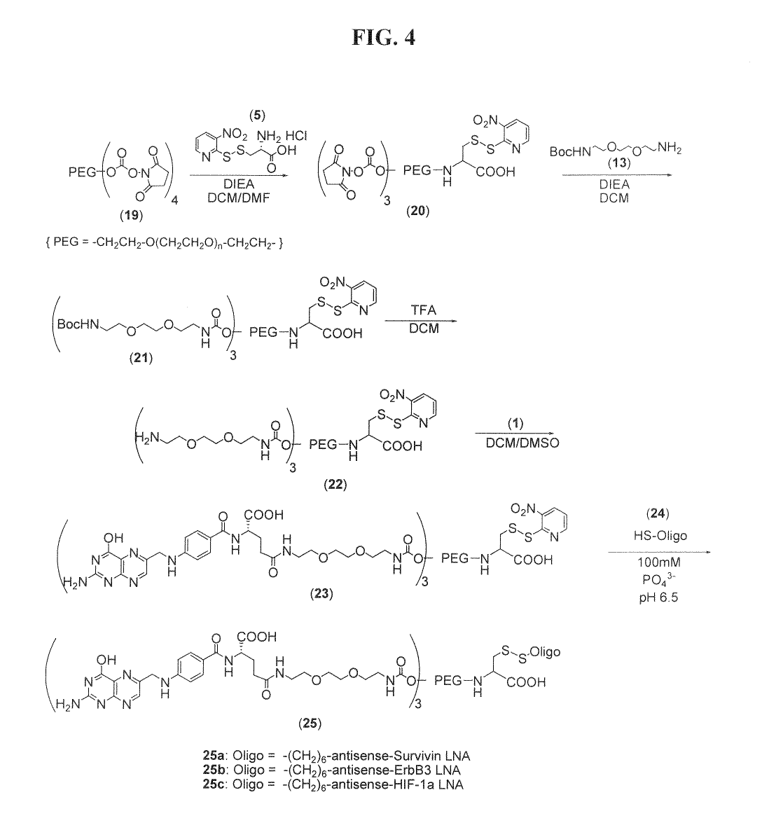

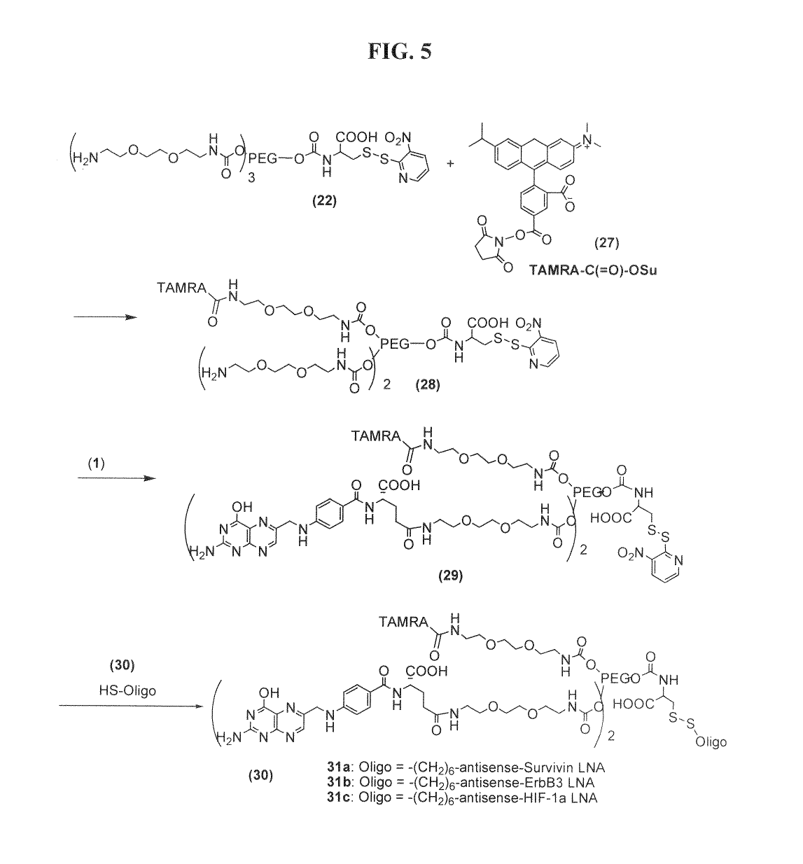

The following examples serve to provide further appreciation of the invention but are not meant in any way to restrict the effective scope of the invention. The bold-faced numbers recited in the Examples correspond to those shown in FIGS. 1-6.

### Example 1

### General Experimentals

All synthesis reactions are run under an atmosphere of dry nitrogen or argon. Commercial reagents are used without further purification. All PEG compounds were dried in vacuo or by azeotropic distillation from toluene prior to use. ¹H NMR spectra were obtained at 300 MHz and ¹³C NMR spectra at 75.46 MHz using a Varian Mercury 300 NMR spectrometer and deuterated chloroform as the solvents unless otherwise specified. Chemical shifts (δ) are reported in parts per million (ppm) downfield from tetramethylsilane (TMS).

Abbreviations are used throughout the examples such as DCM (dichloromethane), DIEA (N,N-Diisopropylethylaamine), LNA (Locked Nucleic Acid), MEM (Modified Eagle's Medium), TEAA (tetraethylammonium acetate), TFA (trifluoroacetic acid), and RT-qPCR (reverse transcription-quantitative polymerase chain reaction).

### Example 2

### General HPLC Method

The reaction mixtures and the purity of intermediates and final products are monitored by a Beckman Coulter System Gold® HPLC instrument. It employs a ZORBAX® 300SB C8 reversed phase column (150×4.6 mm) or a Phenomenex Jupiter® 300A C18 reversed phase column (150×4.6 mm) with a 168 Diode Array UV Detector, using a gradient of 10-90% of acetonitrile in 0.05% TFA at a flow rate of 1 mL/minute or a gradient of 25-35% acetonitrile in 50 mM TEAA buffer at a flow rate of 1 mL/minute. The anion exchange chromatography was run on AKTA explorer 100A from GE healthcare (Amersham Biosciences) using Poros 50HQ strong anion exchange resin from Applied Biosystems packed in an AP-Empty glass column from Waters. Desalting was achieved by using HiPrep 26/10 desalting columns from Amersham Biosciences.

### Example 3

### General mRNA Down-Regulation Procedure

The cells were maintained in complete medium (F-12K or DMEM, supplemented with 10% FBS). A 12 well plate containing 2.5×10⁵ cells in each well was incubated overnight at 37° C. Cells were washed once with Opti-MEM® and 400 μL of Opti-MEM® was added per each well. Then, a solution of the polymer conjugate containing oligonucleotide was added to each well. The cells were incubated for 4 hours, followed by addition of 600 μL of media per well, and incubation for 24 hours. After 24 hours of treatment, the intracellular mRNA levels of the target gene, such as human survivin, and a housekeeping gene, such as GAPDH were quantitated by RT-qPCR. The expression levels of mRNA normalized to that of GAPDH were compared.

### Example 4

### General RNA Preparation Procedure

For the in vitro mRNA down-regulation studies, total RNA was prepared using RNAqueous Kit® (Ambion) following the manufacturer's instruction. The RNA concentrations were determined by OD_{260 nm} using Nanodrop.

### Example 5

### General RT-qPCR Procedure

All the reagents were from Applied Biosystems: High Capacity cDNA Reverse Transcription Kit® (4368813), 20×PCR master mix (4304437), and TaqMan® Gene Expression Assays kits for human GAPDH and survivin. 2.0 μg of total RNA was used for cDNA synthesis in a final volume of 50 μL. The reaction was conducted in a PCR thermocycler at 25° C. for 10 minutes, 37° C. for 120 minutes, 85° C. for 5 seconds and then stored at 4° C. Real-time PCR was conducted with the program of 50° C.-2 minutes, 95° C.-10 minutes, and 95° C.-15 seconds/60° C.-1 minute for 40 cycles. For each qPCR reaction, 1 μL of cDNA was used in a final volume of 30 μL.

### Example 6

### General Procedure for PEGylation of Oligonucleotides

A solution of activated PEG (0.35 mmol, about 10-30 eq) and oligonucleotides (0.03 mmol, ˜1 eq) in PBS buffer (˜5 mL/100 mg PEG, pH 7.4) is stirred at room temperature and the reaction progress is monitored by HPLC. The reaction is diluted in Milli-Q water (25 mL) and purified using a HQ/10 Poros strong anion exchange column (e.g. Source 15RPC column equilibrated with 100 mM TEAA before loading, 10 mm×60 mm, bed volume ˜6 mL). The fractions are eluted using 1M NaCl, water, and 50% CH₃CN. The fractions containing pure product are pooled and lyophilized to yield pure PEG-Oligo. MALDI is used to confirm the molecular weight of the product.

### Example 7

### Preparation of Compound 1 (Folate NHS)

Folic acid was coupled with NHS in the presence of DCC to provide an NHS ester (compound 1).

### Example 8

### Preparation of Compound 3

Heterobifunctional amino acid PEG (compound 2) is coupled with NHS in the presence of EDC to provide an NHS ester (compound 3).

### Example 9

### Preparation of Compound 5

A solution of 4 N HCl in dioxane (70 mL) was added to BocCys(Npys)-OH (compound 4, 5 g, 13.32 mmol). The suspension was stirred at room temperature for 3 hours, and then was poured into 700 mL of ethyl ether. The solid was filtered through a course fritted funnel without applying vacuum until the end. The cake was washed with ethyl ether (3×50 mL) and then dried under vacuum at room temperature overnight. ¹H NMR (300 MHz, CD₃OD): δ 8.93 (1H, dd, J=1.5, 4.7 Hz), 8.66 (1H, dd, J=1.5, 8.20 Hz), 7.59 (1H, dd, J=4.7, 8.2 Hz), 4.24 (1H, dd, J=4.1, 9.4 Hz), 3.58 (1H, dd, J=4.1, 14.9 Hz), 3.36 (1H, dd, J=9.4, 15.2 Hz). ¹³C NMR (75.4 MHz, CDCl₃): δ 169.40, 156.27, 154.64, 144.13, 135.246, 123.10, 52.77, 39.27.

### Example 10

### Preparation of Compound 6

Compound 3 (0.35 mmol) is added to a solution of compound 5 (2 equivalent for each NHS to be substituted) in DMF/DCM (25 mL/45 mL), followed by addition of DIEA (3 equivalent for each compound 5). The suspension is stirred at room temperature for 5 hours. The reaction mixture is evaporated under vacuum and then precipitated with DCM/Et₂O at 0° C. The solid is filtered and then is dissolved in 80 mL of DCM. After addition of 20 mL of 0.1 N HCl, the mixture is stirred for 5 minutes, then transferred to a reparatory funnel and the organic layer is separated and washed again with 0.1 N HCl (20 mL) and brine (20 mL). The organic layer is dried over MgSO₄, filtered and evaporated under vacuum. The residue is precipitated with DCM/Et₂O at 0° C. The solid is filtered and dried in the vacuum oven at 30° C. for at least 2 hours to give compound 14.

### Example 11

### Preparation of Compound 7

A solution of compound 6 in DCM (10 mL/g of compound 6) is added 16 mL TFA (2.5 mL/g of compound 6) at 0° C. The reaction mixture is stirred at 0° C. to room temperature for 1 hour. After completion of reaction, the solvent is removed in vacuo and the residue is precipitated from 20 mL/300 mL/g of compound 6 of DCM/Et₂O at 0° C. Solids are filtered and dried to get compound 7.

### Example 12

### Preparation of Compound 8

A solution of compound 1 in DMSO is added to a solution of compound 7 in DCM. The reaction mixture is stirred and the crude product is precipitated in DCM/Ether. The solid is filtered and dried in vacuo to give compound 8.

### Example 13

### Preparation of Compound 10

Compound 8 is reacted with oligonucleotides (compound 9, HS-5′-(CH₂)₆-Oligonucleotide) in the conditions described in Example 6 to give compound 10.

### Example 14

### Preparation of Compound 12

Compound 11 (bis SC-PEG, 0.35 mmol) is added to a solution of compound 5 (2 equivalent for each NHS to be substituted) in DMF/DCM (25 mL/45 mL), followed by addition of DIEA (3 equivalent for each compound 5). The suspension is stirred at room temperature for 5 hours. The reaction mixture is evaporated under vacuum and then precipitated with DCM/Et₂O at 0° C. The solid is filtered and then is dissolved in 80 mL of DCM. After addition of 20 mL of 0.1 N HCl, the mixture is stirred for 5 minutes, then transferred to a separatory funnel and the organic layer is separated and washed again with 0.1 N HCl (20 mL) and brine (20 mL). The organic layer is dried over MgSO₄, filtered and evaporated under vacuum. The residue was precipitated with DCM/Et₂O at 0° C. The solid is filtered and dried in the vacuum oven at 30° C. for at least 2 hours to give compound 12.

### Example 15

### Preparation of Compound 14

Compound 13 is reacted with compound 12 in the presence of DIEA as the base in DCM to give compound 14.

### Example 16

### Preparation of Compound 15

Compound 14 is treated with TFA in DCM to give compound 15.

### Example 17

### Preparation of Compound 16

A solution of compound 1 in DMSO is added to a solution of compound 15 in DCM. The reaction mixture is stirred and the crude product is precipitated in DCM/Ether. The solid is filtered and dried in vacuo to give compound 16.

### Example 18

### Preparation of Compound 18

Compound 16 is reacted with oligonucleotides (compound 17, HS-5′-(CH₂)₆-Oligonucleotide) in conditions described in Example 6 to give compound 18.

### Example 19

### Preparation of Compound 20 ((SC)₃-PEG-Cys-SS-NPyS)

Compound 19 (4 arm SC-^{20K}PEG, 0.35 mmol) is added to a solution of compound 5 (2 equivalent for each NHS to be substituted) in DMF/DCM (25 mL/45 mL), followed by addition of DIEA (3 equivalent for each compound 5). The suspension is stirred at room temperature for 5 hours. The reaction mixture was evaporated under vacuum and then precipitated with DCM/Et₂O at 0° C. The solid was filtered and then was dissolved in 80 mL of DCM. After addition of 20 mL of 0.1 N HCl, the mixture was stirred for 5 minutes, then transferred to a separatory funnel and the organic layer was separated and washed again with 0.1 N HCl (20 mL) and brine (20 mL). The organic layer was dried over MgSO₄, filtered and evaporated under vacuum. The residue was precipitated with DCM/Et₂O at 0° C. The solid is filtered and dried in the vacuum oven at 30° C. for at least 2 hours to give compound 20.

### Example 20

### Preparation of Compound 21 ((BocNHExtend)₃-PEG-Cys-SS-NPyS)

Compound 20 was reacted with compound 13 in the presence of DIEA as the base in DCM to give compound 21.

### Example 21

### Preparation of Compound 22 ((NHExtend)₃-PEG-Cys-SS-NPyS)

Compound 21 was treated with TFA in DCM to give compound 22.

### Example 22

### Preparation of Compound 23 ((Folate-NHExtend)₃-PEG-Cys-SS-NPyS)

A solution of compound 1 in DMSO was added to a solution of compound 22 in DCM. The reaction mixture is stirred and the crude product is precipitated in DCM/Ether. The solid was filtered and dried in vacuo to give compound 23.

### Example 23

### Preparation of Compound 25 ((Folate-NHExtend)₃-^{20K}PEG-Cys-SS-C₆-Oligo)

(Folate)₃-^{20K}PEG-NPyS (compound 23, 120 mg, 5.50 μmol) was dissolved in pH 6.5 phosphate buffer (3-4 mL), covered in foil and purged with nitrogen gas for 10 minutes. To this solution was added oligonucleotides (compound 24, e.g. antisense ErbB3 LNA oligonucleotide, 6.0 mg, 1.10 μmol) and the resulting orange yellow mixture was stirred for ˜2 hours at ambient temperature during which time the solution became deeper yellow in color. After this time, the solution was filtered using a 0.45 μm syringe filter and loaded on a Poros HQ, strong anion exchange column (10 cm×1.0 cm, bed volume ˜8 mL) which was pre-equilibrated with 20 mM Tris-HCl buffer, pH 7.0 (buffer A). The column was washed with 3-4 column volumes of buffer A to remove the excess PEG linker. The product was eluted by slow incremental gradient of 1M NaCl in 20 mM Tris-HCl buffer, pH 7.0 (buffer B). The isolated fractions were combined and desalted via reverse-phase chromatography (Source RPC) and the resulting solution was lyophilized to yield the desired PEG-LNA compound as a fluffy yellow solid (4.62 mg, based on oligo, 77.0%).

### Example 24

### Preparation of Compound 26 ((Folate-NHExtend)₃-^{40K}PEG-Cys-SS-C₆-Oligo-FAM)

(Folate)₃-^{40K}PEG-NPyS (0.35 g, 8.33 μmol) was dissolved in pH 6.5 phosphate buffer (5 mL), covered in foil and purged with nitrogen gas for 10 minutes. To this solution was added FAM-modified oligonucleotides (FAM modified compound 24, 10.0 mg, 1.67 μmol) and the resulting orange yellow mixture was stirred for ˜3 hours at ambient temperature during which time the solution became deeper yellow in color. After this time, the solution was filtered using a 0.45 μm syringe filter and loaded on a Poros HQ, strong anion exchange column (10 cm×1.0 cm, bed volume ˜8 mL) which was pre-equilibrated with 20 mM Tris-HCl buffer, pH 7.0 (buffer A). The column was washed with 3-4 column volumes of buffer A to remove the excess PEG linker. The product was eluted by slow incremental gradient of 1M NaCl in 20 mM Tris-HCl buffer, pH 7.0 (buffer B). The isolated fractions were combined and desalted via reverse-phase chromatography (Source RPC) and the resulting solution was lyophilized to yield the desired PEG-LNA compound as a fluffy yellow solid (3.33 mg, based on oligo, 33.3%).

### Example 25

### Preparation of Compound 28

Compound 22 is reacted with compound TAMRA-C(═O)—OSu (compound 27) to give compound 28.

### Example 26

### Preparation of Compound 29

A solution of compound 1 in DMSO is added to a solution of compound 29 in DCM. The reaction mixture is stirred and the crude product is precipitated in DCM/Ether. The solid is filtered and dried in vacuo to give compound 29.

### Example 27

### Preparation of Compound 31 (Folate)-2-TAMRA-^{20K}PEG-Cys-SS-C₆-Oligo

Compound 29 is reacted with oligonucleotides (compound 30) in conditions described in Example 6 to give compound 31.

### Example 28

### Preparation of Compound 33

Compound 32 (4 arm SC-^{20K}PEG, 0.35 mmol) was added to a solution of compound 5 (2 equivalent for each NHS to be substituted) in DMF/DCM (25 mL/45 mL), followed by addition of DIEA (3 equivalent for each compound 5). The suspension is stirred at room temperature for 5 hours. The reaction mixture was evaporated under vacuum and then precipitated with DCM/Et₂O at 0° C. The solid was filtered and then was dissolved in 80 mL of DCM. After addition of 20 mL of 0.1 N HCl, the mixture was stirred for 5 minutes, then transferred to a reparatory funnel and the organic layer was separated and washed again with 0.1 N HCl (20 mL) and brine (20 mL). The organic layer was dried over MgSO₄, filtered and evaporated under vacuum. The residue is precipitated with DCM/Et₂O at 0° C. The solid is filtered and dried in the vacuum oven at 30° C. for at least 2 hours. ¹³C NMR (75.4 MHz, CDCl₃): δ 170.90, 156.66, 155.68, 153.86, 142.41, 133.85, 121.24, 72.96-69.30, 64.08, 53.01, 41.82.

### Example 29

### Preparation of Compound 35

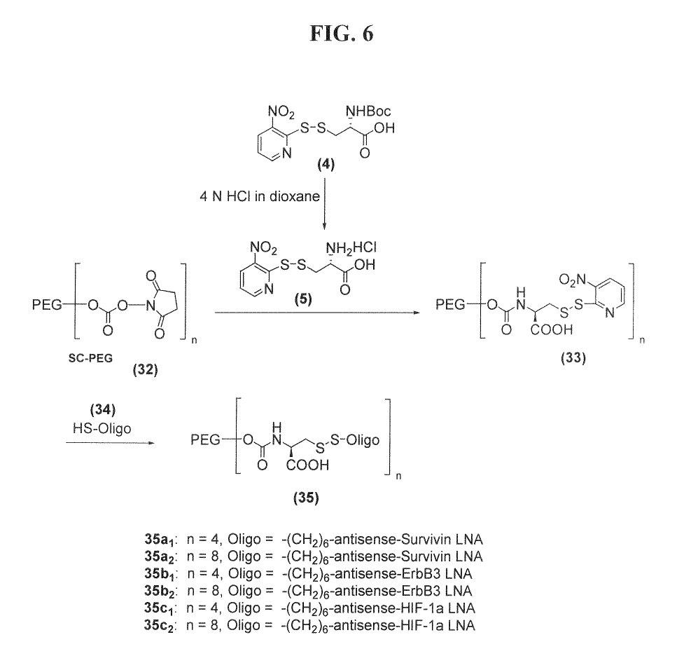

Different sizes of activated PEG polymer were used to make PEG-LNA conjugates. In general, compound 33 was reacted with oligonucleotides (compound 34) using the conditions described in Example 6. The description of each compound is provided in FIG. 6. Oligonucleotides (compound 34, 400 mg, 0.074 mmol) were added to a solution of compound 33 (505 mg, 0.012 mmol) in 25 mL of pH 6.5, 100 mM Sodium Phosphate. The reaction was stirred at room temperature under nitrogen for 5 hours. The reaction mixture was purified on Source 15RPC column. The column was equilibrated with 100 mM TEAA. Then the reaction mixture was loaded. The column was eluted with 1M NaCl, water, and 50% CH₃CN. Compound 35 was collected and lyophilized. Yield 150 mg. MALDI confirms the molecular weight of 62,590.

### Example 30

### Compounds of Formula (I)

Examples 31-36 demonstrate improved tumor delivery of oligonucleotides as well as improved antisense knockdown of targeted tumor mRNA using the releasably linked PEG molecule having the formula:

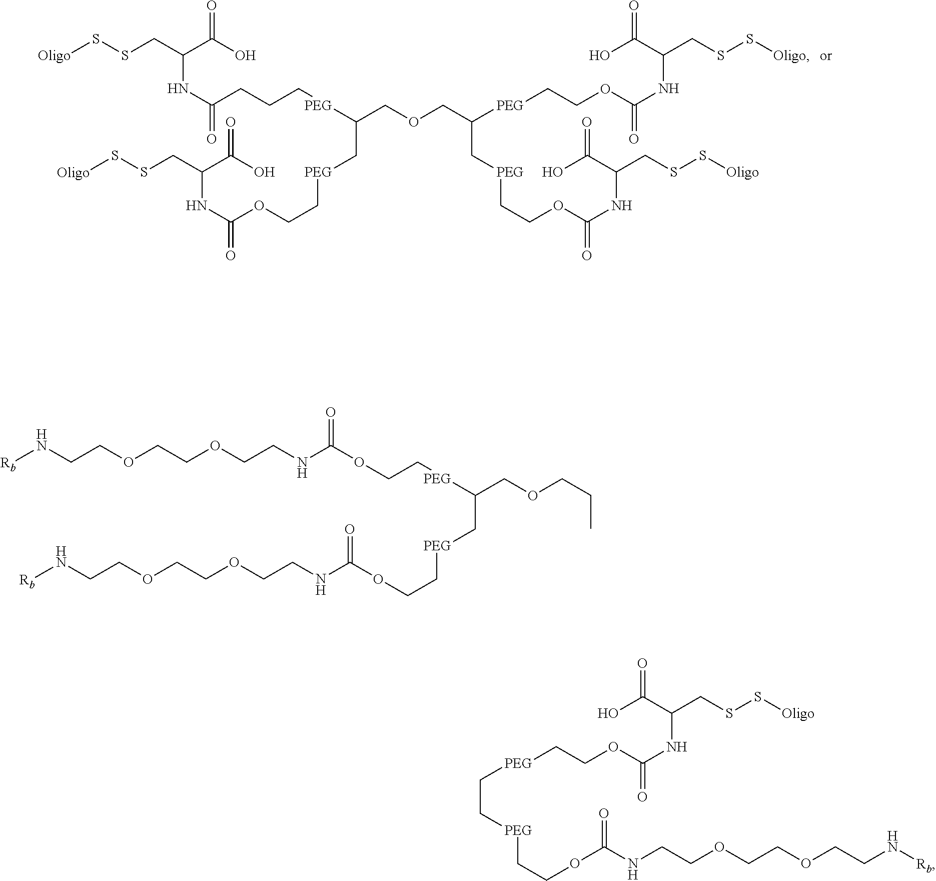

wherein

PEG is a polyethylene glycol;

R_{b} is

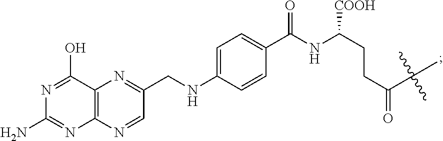

and

Oligo is uniformly 5′-(CH₂)₆-anti-survivin LNA (SEQ ID NO: 1, referred to as “LNA1”) or 5′-(CH₂)₆-anti-erbB3 LNA (SEQ ID NO: 6, referred to “LNA2”), and

the total molecular weight of the polymeric portion of the compound containing PEG is about 40,000 daltons, 20,000 daltons, 10,000 daltons or 5,000 daltons.

For example, the compounds include Folate-^{40K}PEG-Cys-SS-LNA2 (compound 101), Folate-^{5K}PEG-Cys-SS-LNA2 (compound 102), ^{40K}PEG-Cys-SS-LNA1 (compound 103), ^{10K}PEG-Cys-SS-LNA1, (compound 4) and ^{40K}PEG-Cys-SS-LNA2 (compound 105).

### Example 31

### In Vitro Stability

The PEG-Cys-SS-LNA and Folate-PEG-Cys-SS-LNA conjugates described herein showed good stability in buffers.

### Example 32

### In Vitro Cellular Uptake of Folate-PEG-Cys-SS-LNA Conjugate

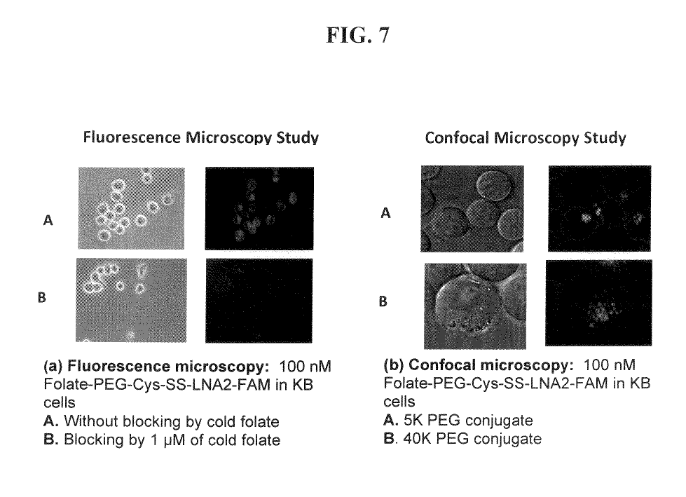

Cellular uptake of Folate-PEG-Cys-SS-LNA conjugate and specificity of binding to the folate receptor were evaluated in KB human cervical carcinoma cell line. KB cells were plated overnight at 37° C. The cells were incubated with 100 nM of Folate-PEG-Cys-SS-LNA2-FAM conjugates (compounds 101 and 102 labeled with FAM) with or without 1 μM of free folate at 37° C. for 24 hours. The amount of the PEG-Cys-SS-LNA2-FAM conjugate was based on the amount of LNA2, not the amount of polymeric conjugate. The cells were washed and the samples were observed under fluorescence microscope and confocal microscope. The results of the cellular uptake of the Folate-PEG-Cys-SS-LNA conjugate are shown in FIG. 7.

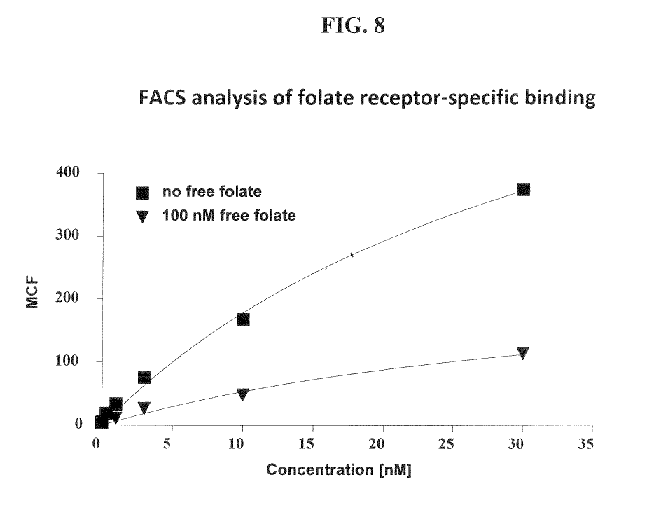

KB cells were also exposed to Folate-^{5K}PEG-Cys-SS-LNA2-FAM (compound 102 labeled with FAM) with or without 100 nM of free folate at 37° C. for 4 hours. The cells were washed and analyzed by FACS for the specificity of binding. The results are shown in FIG. 8.

Both fluorescence and confocal microscope studies showed that folate improved cellular uptake of LNA oligonucleotides. The intracellular delivery of the folate-PEG conjugate was comparable to that transfected with lipofectamine. Folic acid enhanced intracellular uptake of oligonucleotides.

The fluorescence microscope images (FIG. 7(a)) and FACS analysis (FIG. 8) showed that the binding of the folate-PEG conjugate to the folate receptor and subsequent internalization into KB cells was blocked in the presence of free folate. The binding of the folate-PEG conjugate to the folate receptor in KB cells was specific.

### Example 33

### In Vitro Efficacy of PEG-Cys-SS-LNA Conjugate in Tumor Cells

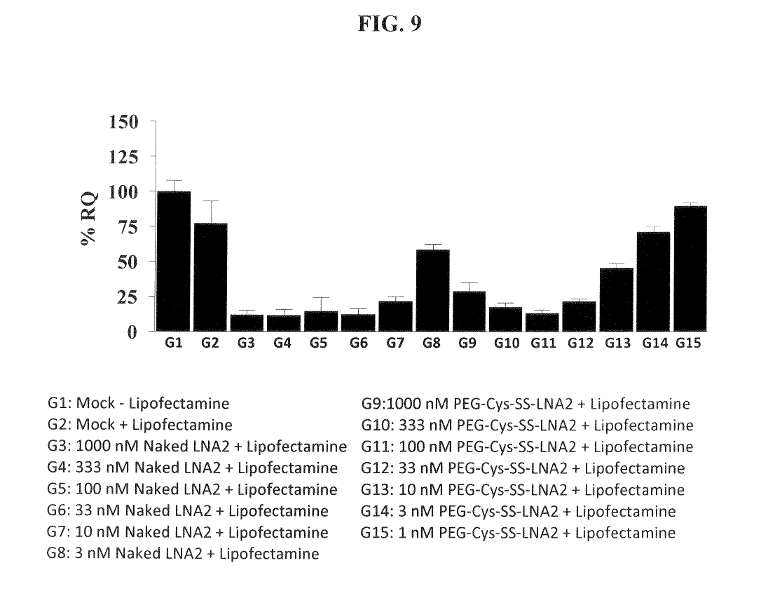

In vitro efficacy of PEG-Cys-SS-LNA conjugates and naked LNA oligonucleotides were performed by using qRT-PCR. 15PC3 human prostate cancer cells were treated with 0.1 nM to 1,000 nM of each of test compounds, naked LNA2 or ^{40K}PEG-Cys-SS-LNA2 (compound 105). The amount of ^{40K}PEG-Cys-SS-LNA2 administered was based on the amount of LNA2, not the amount of polymeric conjugate administered. The cells were collected and analyzed by using qRT-PCR for ErbB3 mRNA downregulation. The results from qRT-PCR were compared to untreated 15PC3 cells with or without lipofectamine. The results are shown in FIG. 9. The results showed that the PEG-Cys-SS-LNA2 conjugate downregulated expression of the target gene ErbB3 mRNA and the efficacy was comparable to naked LNA2. The knockdown of ErbB3 mRNA expression of PEG-Cys-SS-LNA2 conjugate was as potent as equivalent naked LNA2 in 15PC3 cells (IC₅₀=5 nM and 8.5 nM, respectively). The PEG attached to the LNA oligonucleotides did not interfere with the potency of the LNA oligonucleotides.

### Example 34

### In Vivo Efficacy of Folate-PEG-Cys-SS-LNA Conjugate in KB Xenografted Mice Model

The efficacy of Folate-PEG-LNA conjugates was evaluated in KB human cervical cancer xenografted mice. Athymic nude Balb/c mice bearing KB tumor (epidermoid, human cervical carcinoma cell line) were treated with a dose of 35 mg/kg of naked LNA2 or Folate-^{40K}PEG-Cys-SS-LNA2, ^{40K}PEG-LNA2, Folate-^{5K}PEG-LNA2, or ^{5K}PEG-LNA2 conjugate at q3dx4 for 12 days. The amount of the PEG conjugates administered was based on the amount of LNA2, not the amount of polymeric conjugate administered. Tumor and liver samples were isolated and analyzed by using qRT-PCR for ErbB3 mRNA down-regulation.

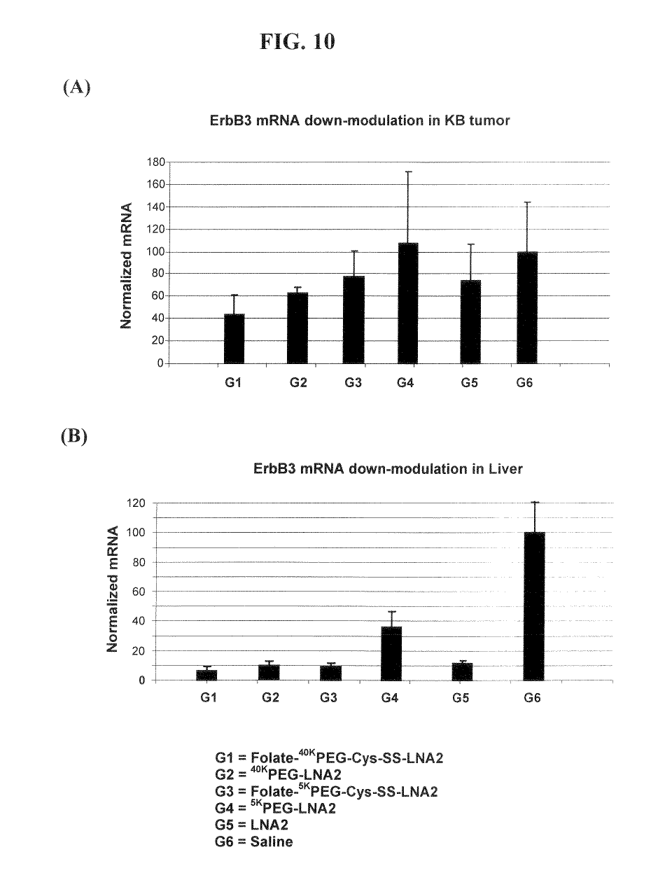

The results are as shown in FIGS. 10(A) and (B). The folate-PEG conjugates significantly inhibited expression of ErbB3 mRNA compared to naked LNA2 in the tumor tissues. Additionally, 40K Folate-PEG-LNA conjugates downregulated target mRNA compared to 5K Folate-PEG-LNA conjugates. The results showed that PEGylation increased accumulation of LNA oligonucleotide in tumor and the folate-PEG conjugates enhanced mRNA downregulation as compared to naked LNA oligonucleotides.

### Example 35

### Biodistribution of PEG-Cys-SS-LNA in Tumor and Plasma

The circulation of PEG-Cys-SS-LNA conjugates in plasma and retention in tumor was evaluated in A549 human long adenocarcinoma xenografted mice.

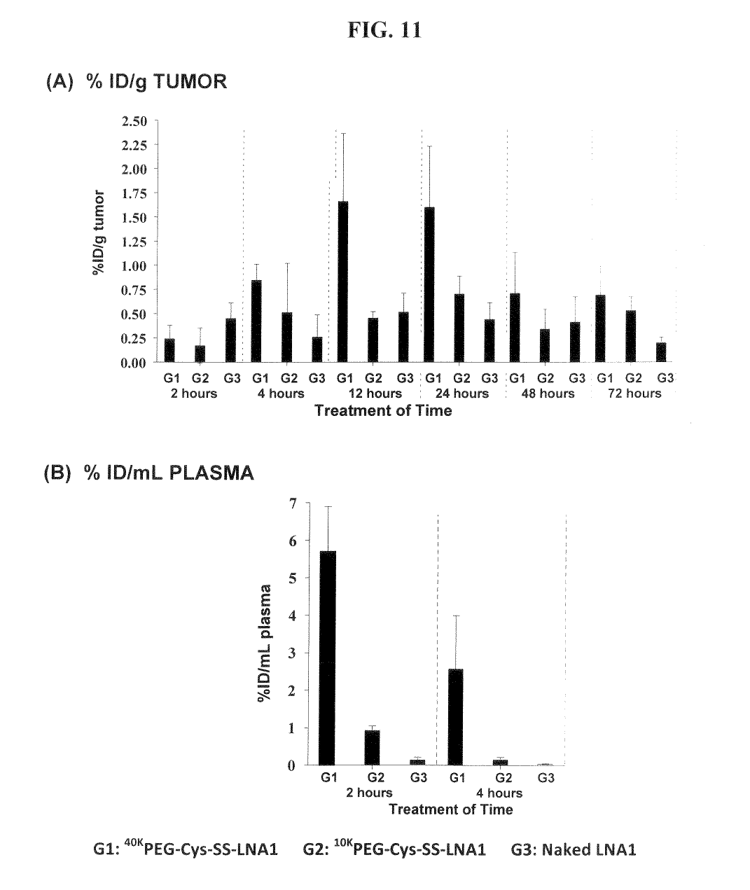

A549 (human lung adenocarcinoma epithelial cell line) cells were implanted sc. in athymic nude mice. When tumor reached the average volume of 75 mm³, the mice were randomly grouped and injected i.v. with a single dose of 10 mg/kg of naked LNA1, ^{40K}PEG-Cys-SS LNA1 (compound 103, 10 mg/kg equivalent dose of LNA1) or ^{10K}PEG-Cys-SS-LNA1 (compound 104, 10 mg/kg equivalent dose of LNA1). Plasma samples were collected at 2 and 4 hours time points following the treatment. Tumor tissues were collected from the sacrificed animals at the various time points (2, 4, 12, 24, 48 and 72 hours) following the treatment. Concentrations of equivalent-LNA1 oligonucleotides in tumor or plasma samples were measured by an ELISA hybridization assay. Results are shown in FIGS. 11(A) and (B).

The results showed that ^{40K}PEG-Cys-SS-LNA conjugate had a significantly higher circulation in plasma and accumulation in tumor compared to naked LNA oligonucleotides. The mice treated with the PEG-Cys-SS-LNA conjugate had >50 times concentration of circulating LNA oligonucleotides in plasma at 2 hours and 4 hours following the treatment, as compared to naked LNA oligonucleotides. The PEG-Cys-SS-LNA conjugates had higher plasma concentrations and longer circulating times compared to naked LNA oligonucleotides. The mice treated with the PEG conjugate had 3-fold higher accumulation of LNA oligonucleotides in tumor at 24 hours, as compared to naked LNA oligonucleotides. The results also indicated that the ^{40K}PEG conjugates had >3.5 times tumor accumulation at 12 hours and maintained ≧1.5 times accumulation up to 72 hours compared to the ^{10K}PEG conjugates. The results indicated that higher molecular weight PEG (40 KDa) conjugates has greater tumor accumulation than lower MW PEG (10 KDa) PEG conjugates.

### Example 36

### In Vivo Efficacy of PEG-Cys-SS-LNA Conjugate in KB Xenografted Mice Model

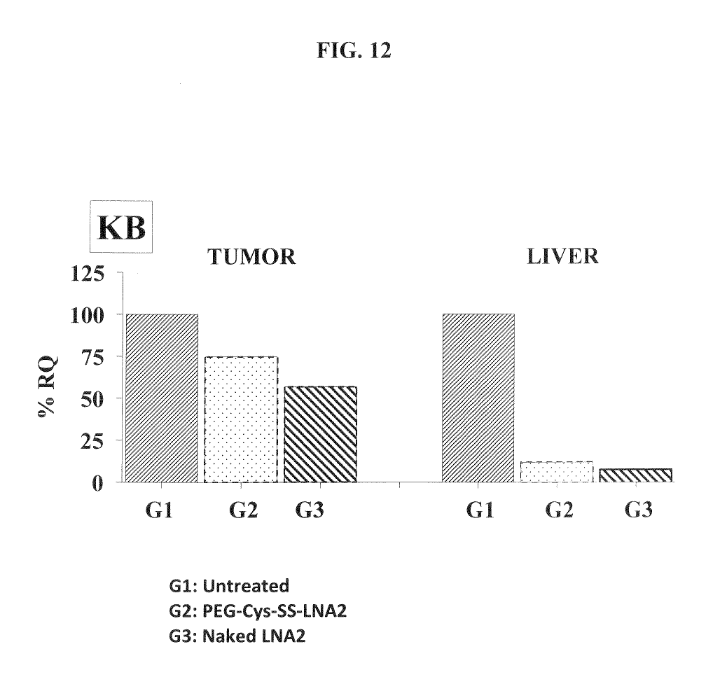

The efficacy of PEG-LNA conjugates was evaluated in KB human cervical cancer xenografted mice. KB cells (epidermoid, human cervical carcinoma cell line) were implanted sc. in nude mice. When tumor reached the average volume of 75 mm³, the mice were randomly grouped and injected i.v. with a single dose of 10 mg/kg of naked LNA2 or ^{40K}PEG-Cys-SS LNA2 (compound 105, 10 mg/kg equivalent dose of LNA2) at q3d x4. Tumor and liver samples were collected 24 hours after the last dose. ErbB3 mRNA downregulation in the samples was measured by using qRT-PCR. The results are shown in FIG. 12.

The results showed that the PEG-Cys-SS-LNA conjugate significantly inhibited expression of ErbB3 mRNA in tumor compared to naked LNA oligonucleotides. The mice treated with the PEG conjugate inhibited ErbB3 mRNA expression 2 fold more than the mice treated with naked LNA. Additionally, the PEG conjugates inhibited 92% ErbB3 mRNA expression in liver as compared to 88% by naked LNA oligonucleotides.

### Example 37

### In Vivo Efficacy of PEG-Cys-SS-LNA Conjugate in 15PC3 Xenografted Mice Model

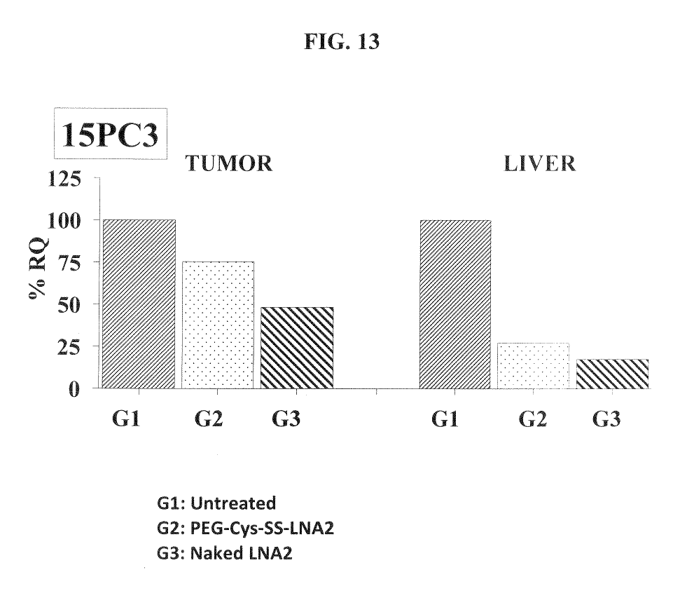

The efficacy of PEG-LNA conjugates was evaluated in 15PC3 human prostate cancer xenografted mice. 15PC3 cells (human prostate cancer cell line) were implanted sc. in nude mice. When tumor reached the average volume of 75 mm³, the mice were randomly grouped and injected i.v. with a single dose of 10 mg/kg of naked LNA2 or ^{40K}(PEG-Cys-SS LNA2 (compound 105, 10 mg/kg equivalent dose of LNA2) at q3d x4. Tumor and liver samples were collected 24 hours after the last dose. ErbB3 mRNA downregulation in the samples was measured by using qRT-PCR. The results are shown in FIG. 13.

The results showed that the PEG-Cys-SS-LNA conjugate significantly inhibited expression of ErbB3 mRNA in tumors compared to naked LNA oligonucleotides. The mice treated with the PEG conjugate inhibited ErbB3 mRNA expression 2 fold more than the mice treated with naked LNA. Additionally, the PEG conjugates inhibited 83% ErbB3 mRNA expression in liver as compared to 73% by naked LNA oligonucleotides.

In view of the above, the invention advantageously provides improved methods employing PEG conjugates for the delivery of oligonucleotides to tumor cells in a mammal that greatly increase circulation time, enhance the accumulation of oligonucleotides in tumors in vivo, while also achieving enhanced downregulation of oncogene mRNA expression in tumors compared to corresponding naked antisense constructs.

One embodiment of the invention provides an improved method for the delivery of oligonucleotides to tumor cells in a mammal that includes the steps of:

(a) providing a compound having the formula:

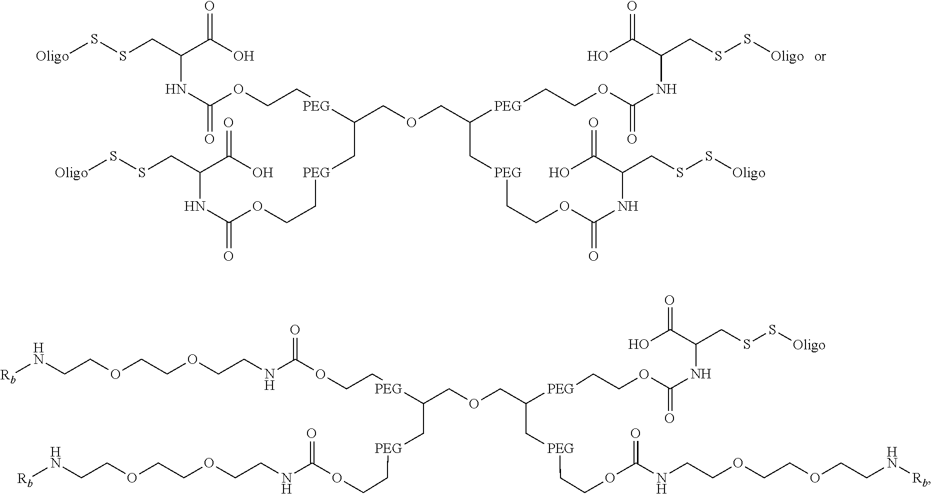

or a pharmaceutically acceptable salt thereof,

wherein

PEG is a polyethylene glycol;

R_{b} is

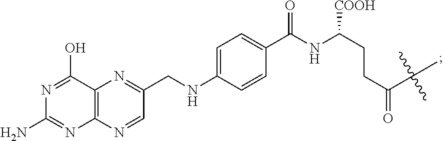

and

Oligo is an oligonucleotide of from about 8 to 30 nucleotides,
wherein the polymeric portion of the compound has the total number average molecular weight of about 40,000 daltons; and

(b) administering the compound or the pharmaceutically acceptable salt thereof to a mammal having tumor cells.

A related embodiment of the invention provides an improved method for the in vivo inhibition of tumor gene expression in a mammal that includes the steps of:

(a) providing a compound having the formula:

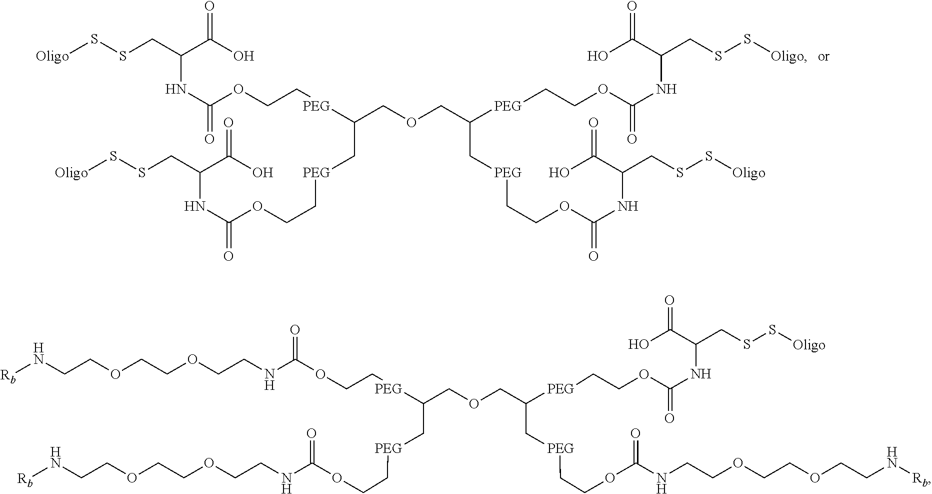

or a pharmaceutically acceptable salt thereof,

wherein

PEG is a polyethylene glycol;

R_{b} is

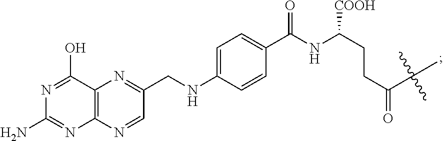

and

Oligo is an oligonucleotide of from about 8 to 30 nucleotides,
wherein the polymeric portion of the compound has the total number average molecular weight of about 40,000 daltons; and

(b) administering the compound or the salt thereof to a mammal having tumor cells, wherein said administration reduces the expression of the preselected gene by the tumor cells.

The inhibition of expression of the preselected gene may be as a result of antisense targeting of an mRNA molecule thereby reducing or eliminating translation of the mRNA to a polypeptide. In the method embodiments above, the administration may be by the blood stream of the mammal, for example, by intravenous (i.v.) injection. The oligonucleotides may comprise LNA. The oligonucleotide includes -5′-(CH₂)₆-antisense-Survivin LNA oligonucleotide or -5′-(CH₂)₆-antisense-ErbB3 LNA oligonucleotide.

We claim:

1. An improved method of delivering oligonucleotides to tumor cells in a mammal, comprising administering to a mammal having tumor cells a compound of Formula (I):
R₁—{Z₁}ₘ
or a pharmaceutically acceptable salt thereof,
wherein
R₁ is a substantially non-antigenic water-soluble polymer;
each Z₁ is the same or different and selected from the group consisting of 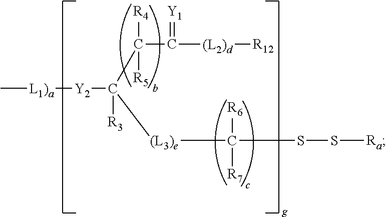
-(L₄)ₐ₁-R_{b}; and
-(L₄)ₐ₂-R_{c},
Y₁, in each occurrence, is independently S or O;
Y₂, in each occurrence, is independently NR₁₃;
Rₐ, in each occurrence, is the same or a different oligonucleotide;
each of L₁₋₄, in each occurrence, is the same or a different bifunctional linker;
R_{b}, in each occurrence, is a folic acid;
R_{c}, in each occurrence, is the same or a different diagnostic agent;
each of R₃₋₇ is independently selected from the group consisting of hydrogen, C₁₋₆ alkyls, C₂₋₆ alkenyl, C₂₋₆ alkynyl, C₃₋₁₉ branched alkyl, C₃₋₈ cycloalkyl, and C₁₋₆ alkoxy;
R₁₃, in each occurrence, is independently selected from the group consisting of hydrogen, C₁₋₆ alkyls, C₂₋₆ alkenyl, C₂₋₆ alkynyl, C₃₋₁₉ branched alkyl, and C₃₋₈ cycloalkyl;
R₁₂, in each occurrence, is independently selected from the group consisting of hydrogen, hydroxyl, C₁₋₆ alkyls, C₂₋₆ alkenyl, C₂₋₆ alkynyl, C₃₋₁₉ branched alkyl, C₃₋₈ cycloalkyl and C₁₋₆ alkoxy;
each of (a) and (d) is independently zero, 1, 2, or 3;
each of (a1) and (a2) is independently zero, 1, 2, or 3;
each (b) is independently zero, 1, 2, or 3;
each (c) is independently zero, 1, 2, or 3;
each (e) is independently zero or one;
each (g) is independently zero or one; and
(m) is a positive integer from about 2 to about 32,
provided that (a) and (g) are not simultaneously zero and further provided that one or more of Z₁ contains an oligonucleotide.

2. The method of claim 1, wherein the compound of Formula (I) has Formula (I′): 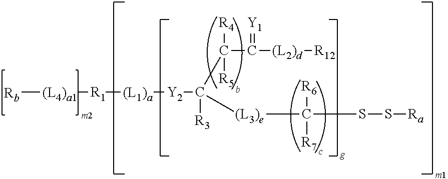
wherein
(m1) is a positive integer from about 1 to about 8;
(m2) is zero or a positive integer from about 1 to about 7; and
the sum of (m1) and (m2) is an integer from about 2 to about 8.

3. The compound of claim 1, wherein all Z₁ contain an oligonucleotide.

4. The method of claim 1, wherein one or more of Z₁ contains a folic acid.

5. The method of claim 1, wherein R₁₂ is OH.

6. The method of claim 1, wherein R₃₋₇ are all hydrogen.

7. The method of claim 1, wherein (b), (d) and (e) are zero, and (c) is one.

8. The method of claim 1, wherein Z₁ has the formula: 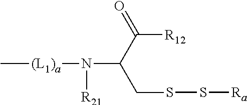
wherein,
(a) is 0 or 1;
(m) is an integer from 2 to 8; and (2, 4, 8, 16 or 32);
R₁₂, in each occurrence, is independently selected from the group consisting of hydroxyl, C₁₋₆ alkyls, C₂₋₆ alkenyl, C₂₋₆ alkynyl, C₃₋₁₉ branched alkyl, and C₁₋₆ alkoxy; and
all other variables are the same as defined in claim 1.

9. The method of claim 2, wherein the compound of Formula (I) has the formula 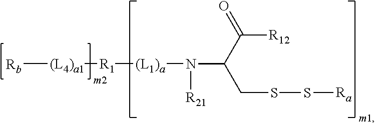
wherein (a) is 0 or 1.

10. The method of claim 9, wherein (m2) is zero.

11. The method of claim 1, wherein (m1) is one.

12. The method of claim 1, wherein R₁ comprises a polyalkylene oxide.

13. The method of claim 12, wherein R₁ has the total number average molecular weight of from about 5,000 to about 25,000 daltons or from about 20,000 to about 45,000 daltons.

14. A compound of claim 1 selected from the group consisting of: 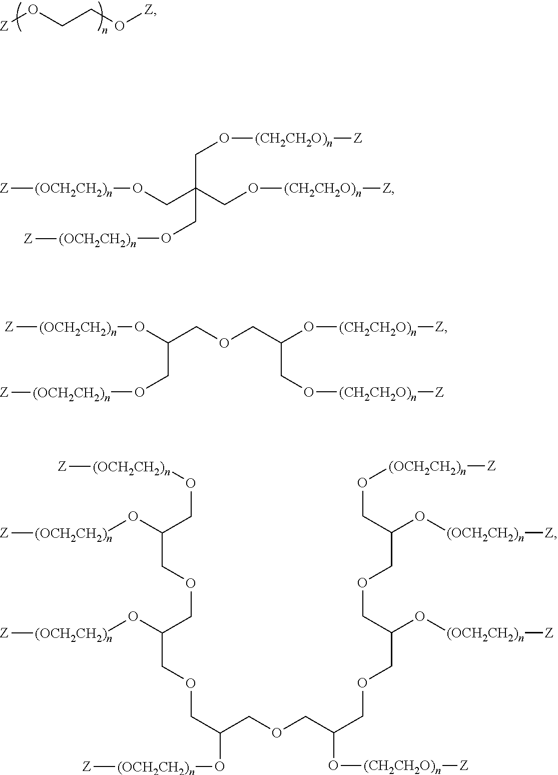
wherein
each Z is independently 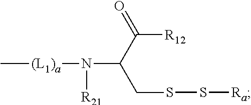
-(L₄)ₐ₁-R_{b}; or
-(L₄)ₐ₂-R_{c},
wherein
(a) is 0 or 1.
R₁₂, in each occurrence, is independently selected from the group consisting of hydroxyl, C₁₋₆ alkyls, C₂₋₆ alkenyl, C₂₋₆ alkynyl, C₃₋₁₉ branched alkyl, and C₁₋₆ alkoxy;
(n) is a positive integer and the polymeric portion of the compound has the total number average molecular weight of from about 5,000 to about 25,000 daltons or from about 20,000 to about 45,000 daltons; and
all other variables are the same as defined in claim 1.

15. The method of claim 1, wherein the oligonucleotide is a single stranded or double stranded oligonucleotide.

16. The method of claim 15, wherein the oligonucleotide is an antisense oligonucleotide.

17. The method of claim 15, wherein the oligonucleotide is selected from the group consisting of deoxynucleotide, ribonucleotide, locked nucleic acids (LNA), short interfering RNA (siRNA), microRNA (miRNA), aptamers, peptide nucleic acid (PNA), phosphorodiamidate morpholino oligonucleotides (PMO), tricyclo-DNA, double stranded oligonucleotide (decoy ODN), catalytic RNA (RNAi), aptamers, spiegelmers, CpG oligomers and combinations thereof.

18. The method of claim 15, wherein the oligonucleotide has LNA and phosphorothioate linkages.

19. The method of claim 15, wherein the oligonucleotide has from about 8 to about 30 nucleotides.

20. The method of claim 19, wherein the oligonucleotide is selected from the group consisting of antisense bcl-2 oligonucleotides, antisense HIF-1α oligonucleotides, antisense survivin oligonucleotides and antisense Erbβ3 oligonucleotides.

21. The method of claim 15, wherein the oligonucleotide comprises SEQ ID NO: 1, SEQ ID NOs 2 and 3, SEQ ID NO:3, SEQ ID NO: 4, SEQ ID NO: 5, SEQ ID NO: 5, and SEQ ID NO: 6.

22. The method of claim 1, wherein the compound of Formula (I) is selected from the group consisting of: 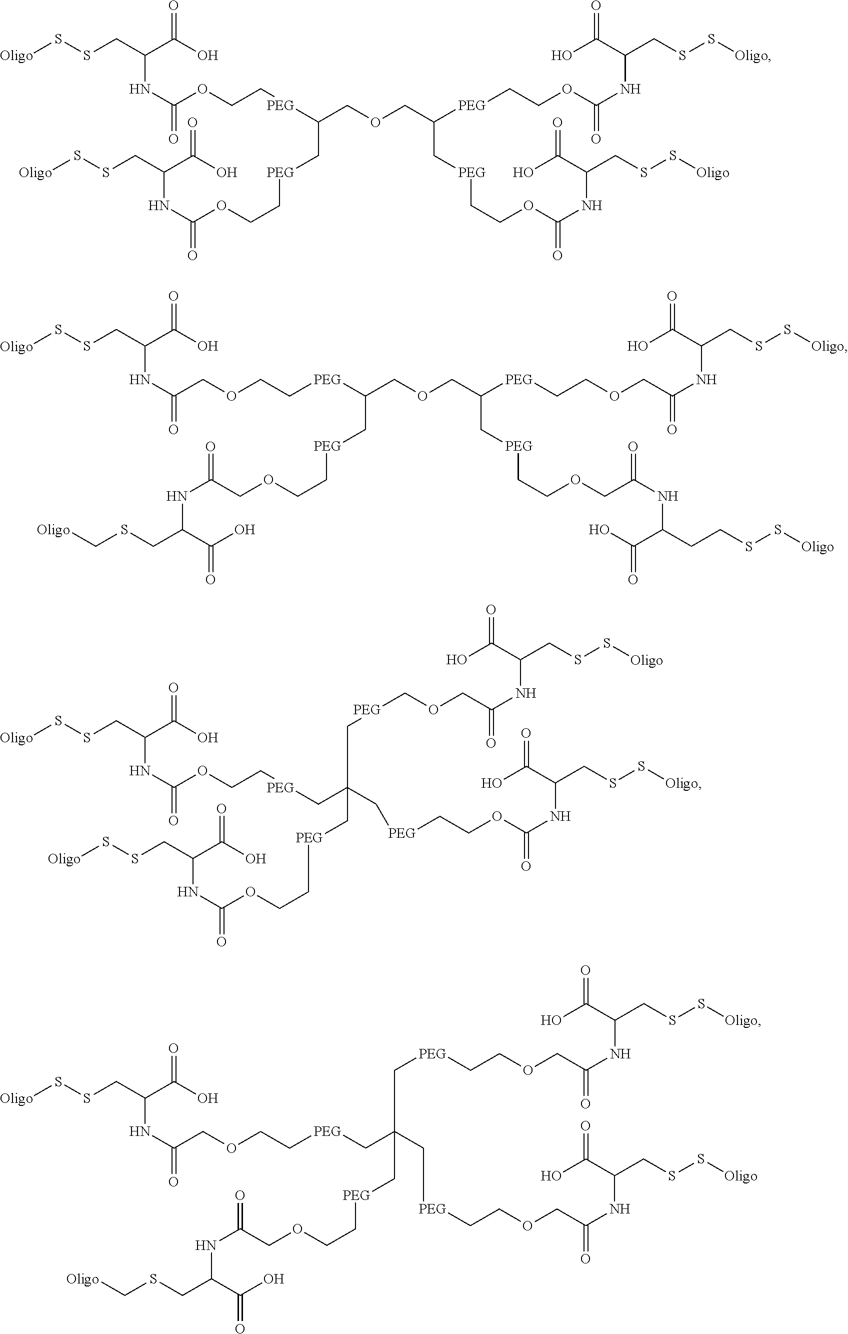 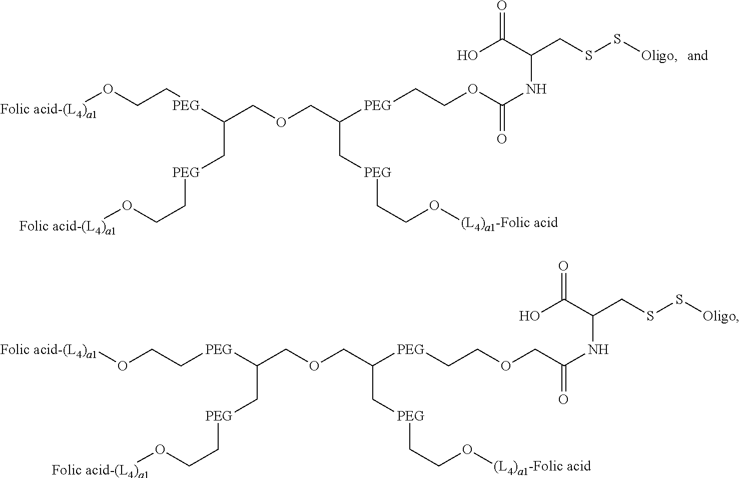
wherein:
Oligo is an oligonucleotide;
PEG is a polyethylene glycol and the polymeric portion of the compound has the total number average molecular weight of from about 5,000 to about 25,000 daltons or from about 20,000 to about 45,000 daltons;
(a1) is one; and
L₄ is —NH(CH₂CH₂O)₂(CH₂)₂NH[C(═O)]ᵣ— or —NH(CH₂)₃—, wherein (r′) is zero or one.

23. The method of claim 1, wherein the tumor cells are prostate or cervical cancer cells.

24. The method of claim 1, wherein the administering step comprises administration via the blood stream of the mammal.

25. An improved method for delivering oligonucleotides to tumor cells in a mammal, comprising:
(a) providing a compound having the formula: 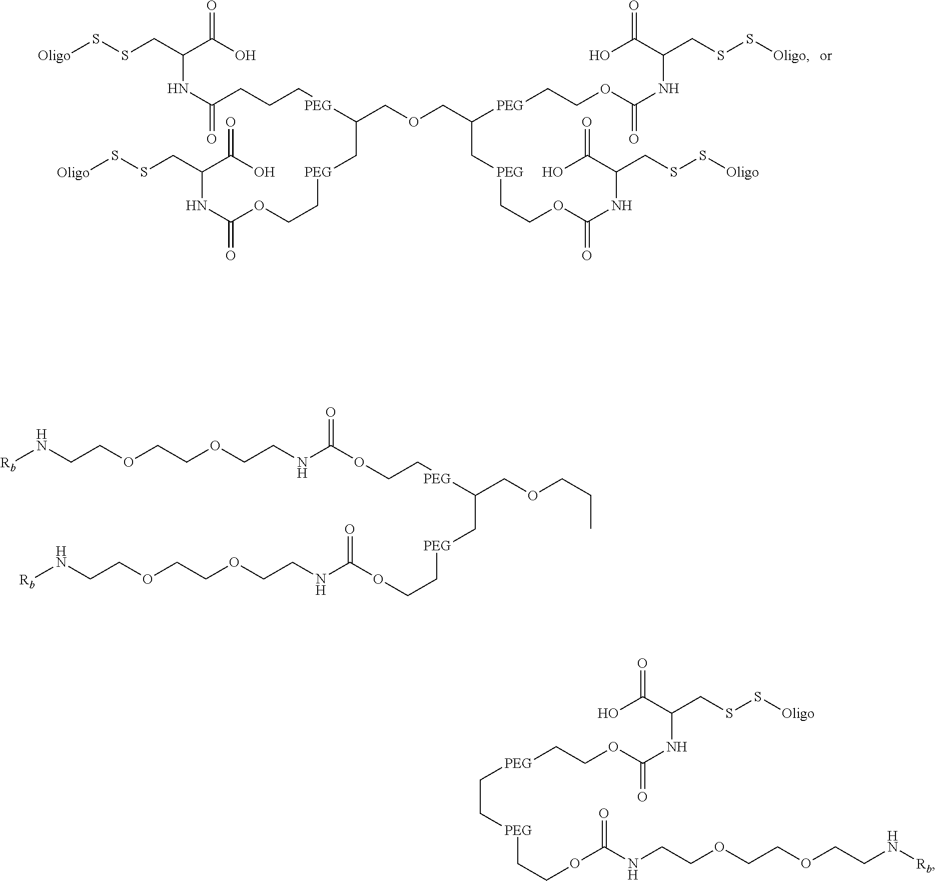
or a pharmaceutically acceptable salt thereof,
wherein
PEG is a polyethylene glycol;
R_{b} is 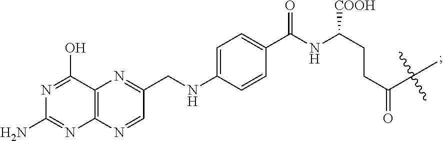
and
Oligo is an oligonucleotide of from about 8 to 30 nucleotides,
wherein the polymeric portion of the compound has the total number average molecular weight of about 40,000 daltons; and
(b) administering the compound or the pharmaceutically acceptable salt thereof to a mammal having tumor cells.

26. The method of claim 25, wherein the oligonucleotide comprises LNA.

27. The method of claim 25, wherein Oligo is -5′-(CH₂)₆-TsAsGsCsCsTsGsTs CsAsCsTsTsCsTsCs-3′ or -5′-(CH₂)₆-GsCsTsGsCsCsAsTsGsGsAsTsTsGsAsG-3′, wherein the first three nucleotides in 5′ and 3′ terminal are LNA and “s” represents a phosphorothioate linkage.

28. The method of claim 25, wherein the tumor cells are prostate or cervical cancer cells.

29. An improved method for delivering oligonucleotides to tumor cells in a mammal, comprising:
(a) providing a compound having the formula: 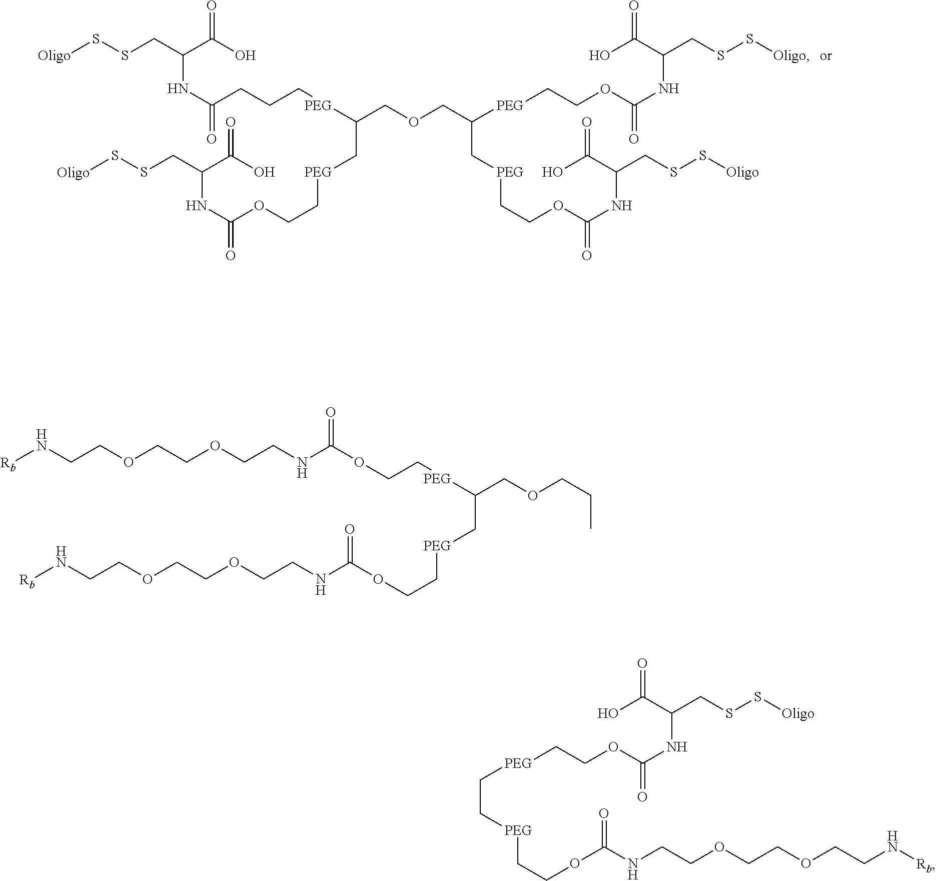
or a pharmaceutically acceptable salt thereof,
wherein
PEG is a polyethylene glycol;
R_{b} is 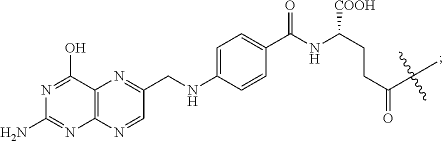
and
Oligo is an oligonucleotide of from about 8 to 30 nucleotides,
wherein the polymeric portion of the compound has the total number average molecular weight of about 40,000 daltons; and
(b) administering the compound or the salt thereof to a mammal having tumor cells, wherein said administration reduces the expression of the preselected gene by the tumor cells.

30. A method of introducing an oligonucleotide into a cell comprising:
contacting a cell with a compound of Formula (I).

31. A method of inhibiting the growth or proliferation of cancer cells comprising:
contacting a cancer cell with a compound of Formula (I).

32. A compound of Formula (Ia):
R₁—{Z₁}ₘ
or a pharmaceutically acceptable salt thereof,
wherein
R₁ is a substantially non-antigenic water-soluble polymer;
each Z₁ is the same or different and selected from the group among 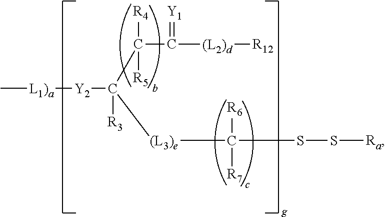
-(L₄)ₐ₁-R_{b}; and
-(L₄)ₐ₂-R_{c},
Y₁, in each occurrence, is independently S or O;
Y₂, in each occurrence, is independently NR₁₃;
Rₐ, in each occurrence, is the same or a different oligonucleotide;
each of L₁₋₄, in each occurrence, is the same or a different bifunctional linker;
R_{b}, in each occurrence, is a folic acid;
R_{c}, in each occurrence, is the same or a different diagnostic agent;
each of R₃₋₇ is independently selected from among hydrogen, C₁₋₆ alkyls, C₂₋₆ alkenyl, C₂₋₆ alkynyl, C₃₋₁₉ branched alkyl, C₃₋₈ cycloalkyl, and C₁₋₆ alkoxy;
R₁₃, in each occurrence, is independently selected from among hydrogen, C₁₋₆ alkyls, C₂₋₆ alkenyl, C₂₋₆ alkynyl, C₃₋₁₉ branched alkyl, and C₃₋₈ cycloalkyl;
R₁₂, in each occurrence, is independently selected from among hydrogen, hydroxyl, C₁₋₆ alkyls, C₂₋₆ alkenyl, C₂₋₆ alkynyl, C₃₋₁₉ branched alkyl, C₃₋₈ cycloalkyl and C₁₋₆ alkoxy;
each of (a) and (d) is independently zero, 1, 2, or 3;
each of (a1) and (a2) is independently zero, 1, 2, or 3;
each (b) is independently zero, 1, 2, or 3;
each (c) is independently zero, 1, 2, or 3;
each (e) is independently zero or one;
each (g) is independently zero or one; and
(m) is a positive integer from about 2 to about 32,
provided that (a) and (g) are not simultaneously zero, and further provided that one or more of Z₁ contain an oligonucleotide, and further provided that one or more of Z₁ contain a folic acid.


SEQUENCE LISTING (6 sequences)

SEQ ID NO: 1 - DNA, 16 bp, Artificial Sequence
  Description of Artificial Sequence Synthetic oligonucleotide
ctcaatccatggcagc

SEQ ID NO: 2 - DNA, 21 bp, Artificial Sequence
  Description of Combined DNA/RNA Molecule Synthetic oligonucleotide
gcaugcggccucuguuugatt

SEQ ID NO: 3 - DNA, 21 bp, Artificial Sequence
  Description of Combined DNA/RNA Molecule Synthetic oligonucleotide
ucaaacagaggccgcaugctt

SEQ ID NO: 4 - DNA, 18 bp, Artificial Sequence
  Description of Artificial Sequence Synthetic oligonucleotide
tctcccagcgtgcgccat

SEQ ID NO: 5 - DNA, 16 bp, Artificial Sequence
  Description of Artificial Sequence Synthetic oligonucleotide
tggcaagcatcctgta

SEQ ID NO: 6 - DNA, 16 bp, Artificial Sequence
  Description of Artificial Sequence Synthetic oligonucleotide
tagcctgtcacttctc